(19) United States
(12) Patent Application Publication
(10) Pub. No.: US 2021/0389323 A1
(43) Pub. Date: Dec. 16, 2021

(54) Methods for the Diagnosis and Treatment of Biofilm-Related Infections

(71) Applicant: Laboratory Corporation of America Holdings, Burlington, NC (US)

(72) Inventors: Stephen Erickson, White Bear Township, MN (US); Jose Gil, Winnetka, CA (US); Matthew Brown, Burlington, NC (US)

(21) Appl. No.: 17/348,706

(22) Filed: Jun. 15, 2021

Related U.S. Application Data
(60) Provisional application No. 63/039,146, filed on Jun. 15, 2020.

(51) Int. Cl. G01N 33/569; A61K 45/06; A61K 35/76; C12N 7/00; A61L 15/44

(52) U.S. Cl. CPC G01N 33/56983; A61K 45/06; C12N 2795/10032; C12N 7/00; A61L 15/44; A61K 35/76

(57) ABSTRACT

Disclosed herein are methods and systems for rapid diagnosis and treatment of biofilm-related infections in a subject having a medical implant. A reporter cocktail composition is disclosed herein and may be used to detect a microorganism of interest and determine the presence of an infection. A therapeutic cocktail composition is also disclosed herein and may be used to treat a subject diagnosed with a biofilm-related infection.

### CROSS REFERENCE TO RELATED APPLICATION

This application claims priority to and the benefit of U.S. Provisional Application No. 63/039,146 filed Jun. 15, 2020, which is incorporated herein by reference in its entirety.

### FIELD OF THE INVENTION

This invention relates to compositions, methods, and systems for the detection and treatment of bacterial infections using infectious agents.

### BACKGROUND

Post-operative infections of implantable devices are a major concern within the healthcare field. These infections can be difficult to diagnose and treat. One particularly complicating factor is the formation of biofilms. Biofilms are a layer of bacteria or other microbes and can be formed from one or more species of bacteria. The bacteria growing as a biofilm reside within a matrix of extracellular polymeric secretions (EPS), which consists of proteins, polysaccharides, and nucleic acids, and allows the biofilm to adhere to natural surfaces, as wells as medical devices. Bacteria secrete EPS into their environment and establish functional and structural integrity of biofilms. Biofilms allow bacteria to share their nutrients and protects the bacteria from harmful factors, including antibiotics.

Biofilms can cause chronic, nosocomial, and medical device-related infections. Such infections are difficult to treat due to their antibiotic-resistant nature, and thus, the use of antibiotics alone is typically ineffective for treating biofilm-related infections (Khatoon et al., 2018, Heliyon). Not only are biofilm-related infections difficult to treat, but they also present challenges for establishing an accurate diagnosis with speciation/sensitivity of the infection.

Antibiotics are widely used to treat infections caused by microorganisms. Some microorganisms are naturally resistant to a particular antibiotic, while others may acquire such resistance after being treated with the antibiotic for some time. Antibiotic resistance can cause undesired consequences; microorganisms still grow in the presence of the antibiotic, therefore exacerbating the infection, and the ineffective antibiotic may cause serious side effects, leading to circumstances, which can be life-threatening in some cases. As a result, antibiotic resistance can lead to higher medical costs, prolonged hospital stays, and increased mortality.

Accordingly, detection of microorganisms that are resistant to particular antibiotics is of great importance. The ability of healthcare providers to determine whether microorganisms responsible for an infection present in the body are resistant to antibiotics is extremely important in selecting the correct treatment. Further, being able to determine the antibiotic resistance of microorganisms within a short timeframe from samples with low levels of microorganisms is vital to successful treatment of infections before they become severe.

Current methods of antibiotic resistance detection, often require assays that are time-consuming, technically-demanding, and/or lack sufficient sensitivity. Typically, these assays involve immunoassays and molecular-based assays in cultured samples that require gel electrophoresis, real time PCR/multiplexing, and/or multi-locus sequence typing. These tests often require 24-48 hours to complete and/or lack sufficient sensitivity. Methods currently available typically require isolation and/or enrichment by culturing of microorganisms prior to detection, thus, requiring increased time-to-results. Therefore, there is a strong interest in a rapid and sensitive test to determine whether a microorganism of interest, e.g., a microorganism that caused an infection, is resistant to a particular antibiotic before using the antibiotic. The present invention excels as a rapid test for the detection of microorganisms by not requiring isolation of the microorganisms prior to detection. This knowledge can aid clinicians in prescribing suitable antibiotics to timely control infections and increase the ability to prevent the spread of serious infections through active monitoring in healthcare settings.

Bacteriophages have been suggested as a replacement or as a supplement to antibiotic treatment. Modifying bacteriophages to optimize enzymatic functions such that they are able to effectively treat biofilm-related infections would be advantageous.

### SUMMARY

Embodiments of the invention comprise compositions, methods, and systems for the diagnosis and treatment of microorganism-related infections. The invention may be embodied in a variety of ways.

In a first aspect of the present disclosure is a method for the diagnosis and treatment of a biofilm-related infection in a subject comprising the steps of: (i) providing a biological sample taken from the subject; (ii) diagnosing the subject with a biofilm-related infection by detecting the presence of at least one microorganism of interest comprising the steps of: (a) contacting at least one aliquot of the biological sample with an amount of a diagnostic cocktail composition comprising at least one recombinant bacteriophage; (b) detecting a signal produced following replication of the recombinant bacteriophage, wherein detection of the signal indicates the microorganism of interest is present in the sample; and (iii) treating the subject diagnosed with a biofilm-related infection comprising the steps of: (a) selecting a therapeutic cocktail composition based on the diagnosis of step (ii); (b) administering a therapeutically-effective amount of a therapeutic cocktail composition comprising at least one bacteriophage, wherein the bacteriophage is specific for the detected microorganism of interest; and (c) optionally administering at least one additional therapeutic agent. In some embodiments the therapeutic cocktail composition comprises at least one bacteriophage, wherein the bacteriophage is a recombinant bacteriophage or a wild-type bacteriophage. In some embodiments, the subject has an implant.

In a second aspect of the present disclosure is a method of preventing or inhibiting infection in a subject comprising applying a cocktail composition comprising at least one recombinant bacteriophage to a surgical implant, dressing, or suture.

In a third aspect of the present disclosure is a surgical implant, dressing, or suture coated in a cocktail composition comprising at least one recombinant bacteriophage.

Certain specific embodiments of the present disclosure make use of methods and constructs described in U.S. Patent Publication No. 2015/0218616 and U.S. Patent Publication No. US 2019/0010534, which are incorporated by reference herein in their entirety.

### DETAILED DESCRIPTION OF THE INVENTION

Disclosed herein are compositions, methods and systems that demonstrate surprising speed and sensitivity for diagnosing bacterial infections and increased treatment efficacy. Diagnosis can be achieved in a shorter timeframe than with currently available methods. The present disclosure describes the use of genetically modified infectious agents in assays.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. Known methods and techniques are generally performed according to conventional methods well known in the art and as described in various general and more specific references that are discussed throughout the present specification unless otherwise indicated. Enzymatic reactions and purification techniques are performed according to manufacturer's specifications, as commonly accomplished in the art or as described herein. The nomenclatures used in connection with the laboratory procedures and techniques described herein are those well-known and commonly used in the art.

The following terms, unless otherwise indicated, shall be understood to have the following meanings:

As used herein, the terms “a”, “an”, and “the” can refer to one or more unless specifically noted otherwise.

The use of the term “or” is used to mean “and/or” unless explicitly indicated to refer to alternatives only or the alternatives are mutually exclusive, although the disclosure supports a definition that refers to only alternatives and “and/or.” As used herein “another” can mean at least a second or more.

Throughout this application, the term “about” is used to indicate that a value includes the inherent variation of error for the device, the method being employed to determine the value, or the variation that exists among samples.

The term “solid support” or “support” means a structure that provides a substrate and/or surface onto which biomolecules may be bound. For example, a solid support may be an assay well (i.e., such as a microtiter plate or multi-well plate), or the solid support may be a location on a filter, an array, or a mobile support, such as a bead or a membrane (e.g., a filter plate, latex particles, paramagnetic particles, or lateral flow strip).

The term “binding agent” refers to a molecule that can specifically and selectively bind to a second (i.e., different) molecule of interest. The interaction may be non-covalent, for example, as a result of hydrogen bonding, van der Waals interactions, or electrostatic or hydrophobic interactions, or it may be covalent. The term “soluble binding agent” refers to a binding agent that is not associated with (i.e., covalently or non-covalently bound) to a solid support.

As used herein, an “analyte” refers to a molecule, compound or cell that is being measured. The analyte of interest may, in certain embodiments, interact with a binding agent. As described herein, the term “analyte” may refer to a protein or peptide of interest. An analyte may be an agonist, an antagonist, or a modulator. Or, an analyte may not have a biological effect. Analytes may include small molecules, sugars, oligosaccharides, lipids, peptides, peptidomimetics, organic compounds and the like.

The term “indicator moiety” or “detectable biomolecule” or “reporter” or “indicator protein product” refers to a molecule that can be measured in a quantitative assay. For example, an indicator moiety may comprise an enzyme that may be used to convert a substrate to a product that can be measured. An indicator moiety may be an enzyme that catalyzes a reaction that generates bioluminescent emissions (e.g., luciferase). Or, an indicator moiety may be a radioisotope that can be quantified. Or, an indicator moiety may be a fluorophore. Or, other detectable molecules may be used.

As used herein, “bacteriophage” or “phage” includes one or more of a plurality of bacterial viruses. In this disclosure, the terms “bacteriophage” and “phage” include viruses such as mycobacteriophage (such as for TB and paraTB), mycophage (such as for fungi), mycoplasma phage, and any other term that refers to a virus that can invade living bacteria, fungi, mycoplasma, protozoa, yeasts, and other microscopic living organisms and uses them to replicate itself. Here, “microscopic” means that the largest dimension is one millimeter or less. Bacteriophages are viruses that have evolved in nature to use bacteria as a means of replicating themselves. A phage does this by attaching itself to a bacterium and injecting its DNA (or RNA) into that bacterium, and inducing it to replicate the phage hundreds or even thousands of times. This is referred to as phage amplification.

As used herein, “late gene region” refers to a region of a viral genome that is transcribed late in the viral life cycle. The late gene region typically includes the most abundantly expressed genes (e.g., structural proteins assembled into the bacteriophage particle). Late genes are synonymous with class III genes and include genes with structure and assembly functions. For example, the late genes (synonymous with class III,) are transcribed in phage T7, e.g., from 8 minutes after infection until lysis, class I (e.g., RNA polymerase) is early from 4-8 minutes, and class II from 6-15 minutes, so there is overlap in timing of II and III. A late promoter is one that is naturally located and active in such a late gene region.

As used herein, “culturing for enrichment” refers to traditional culturing, such as incubation in media favorable to propagation of microorganisms, and should not be confused with other possible uses of the word “enrichment,” such as enrichment by removing the liquid component of a sample to concentrate the microorganism contained therein, or other forms of enrichment that do not include traditional facilitation of microorganism propagation. Culturing for enrichment for periods of time may be employed in some embodiments of methods described herein.

As used herein “recombinant” refers to genetic (i.e., nucleic acid) modifications as usually performed in a laboratory to bring together genetic material that would not otherwise be found. This term is used interchangeably with the term “modified” herein.

As used herein “RLU” refers to relative light units as measured by a luminometer (e.g., GLOMAX® 96) or similar instrument that detects light. For example, the detection of the reaction between luciferase and appropriate substrate (e.g., NANOLUC® with NANO-GLO®) is often reported in RLU detected.

As used herein “time to results” refers to the total amount of time from beginning of sample incubation to generated result. Time to results does not include any confirmatory testing time. Data collection can be done at any time after a result has been generated.

### Samples

Each of the embodiments of the methods and systems of the disclosure can allow for the rapid and sensitive diagnosis and treatment of biofilm-related infections. For example, methods according to the present disclosure can be performed in a shortened time period with superior results.

Microorganisms of interest detectable by the present disclosure include, but are not limited to, bacterial cells that are present in biological samples. In some embodiments, the biological sample may be debrided tissue, blood, serum, plasma, mucosa-associated lymphoid tissue, articular liquid, pleural liquid, saliva, and urine. In some embodiments, irrigation is used to collect biological samples. Irrigation is the flow of a solution (e.g., saline) across an open wound or implanted prosthetic. Thus in some embodiments, the biological sample is a wound irrigant or prosthetic irrigant.

Samples may be liquid, solid, or semi-solid. Samples may be swabs of solid surfaces (e.g., medical implants). In other embodiments the samples may be taken from biological fluid surrounding medical implants. Medical implants include, but are not limited to central venous catheters, heart valves, ventricular assist devices, coronary stents, neurosurgical ventricular shunts, implantable neurological stimulators, arthro-prostheses, fracture-fixation devices, inflatable penile implants, breast implants, cochlear implants, intraocular lenses, dental implants.

In some embodiments, samples may be used directly in the detection methods of the present disclosure, without preparation, concentration, dilution, purification, or isolation. For example, liquid samples, including but not limited to, biological fluids may be assayed directly. Samples may be diluted or suspended in solution, which may include, but is not limited to, a buffered solution or a bacterial culture medium. A sample that is a solid or semi-solid may be suspending in a liquid by mincing, mixing or macerating the solid in the liquid. A sample should be maintained within a pH range that promotes bacteriophage attachment to the host bacterial cell. A sample should also contain the appropriate concentrations of divalent and monovalent cations, including but not limited to Na⁺, Mg²⁺, and Ca²⁺. Preferably a sample is maintained at a temperature that maintains the viability of any pathogen cells contained within the sample.

In some embodiments of the detection assay, the sample is maintained at a temperature that maintains the viability of any pathogen cell present in the sample. For example, during steps in which bacteriophages are attaching to bacterial cells, it is preferable to maintain the sample at a temperature that facilitates bacteriophage attachment. During steps in which bacteriophages are replicating within an infected bacterial cell or lysing such an infected cell, it is preferable to maintain the sample at a temperature that promotes bacteriophage replication and lysis of the host. Such temperatures are at least about 25 degrees Celsius (C), more preferably no greater than about 45 degrees C., most preferably about 37 degrees C.

Assays may include various appropriate control samples. For example, control samples containing no bacteriophages or control samples containing bacteriophages without bacteria may be assayed as controls for background signal levels.

### Indicator Recombinant Bacteriophage

As described in more detail herein, the compositions, methods, and, systems of the disclosure may comprise infectious agents for use in diagnosis of biofilm-related infections. In certain embodiments, the disclosure may include a composition comprising a recombinant indicator bacteriophage, wherein the bacteriophage genome is genetically modified to include an indicator or reporter gene.

A recombinant indicator bacteriophage can include a genetic construct comprising a reporter or indicator gene. In certain embodiments of the recombinant indicator bacteriophage, the indicator gene does not encode a fusion protein. For example, in certain embodiments, expression of the indicator gene during bacteriophage replication following infection of a host bacterium results in a soluble indicator protein product. In some instances, the genetic construct may further comprise an exogenous promoter. In certain embodiments, the genetic construct may be inserted into a late gene region of the bacteriophage. Late genes are generally expressed at higher levels than other phage genes, as they code for structural proteins. The late gene region may be a class III gene region and may include a gene for a major capsid protein.

Some embodiments include designing (and optionally preparing) a sequence for homologous recombination downstream of the major capsid protein gene. Other embodiments include designing (and optionally preparing) a sequence for homologous recombination upstream of the major capsid protein gene. In some embodiments, the sequence comprises a codon-optimized reporter gene preceded by an untranslated region. The untranslated region may include a phage late gene promoter and ribosomal entry site.

In some embodiments of the recombinant indicator phage, the additional, exogenous late promoter (class III promoter, e.g., from phage K or T7 or T4) has high affinity for RNA polymerase of the same native phage (e.g., phage K or T7 or T4, respectively) that transcribes genes for structural proteins assembled into the phage particle. These proteins are the most abundant proteins made by the phage, as each phage particle comprises dozens or hundreds of copies of these molecules. The use of a viral late promoter can ensure optimally high level of expression of the indicator protein product. The use of a late viral promoter derived from, specific to, or active under the original wild-type phage the indicator phage is derived from (e.g., the phage K or T4 or T7 late promoter with a phage K- or T4- or T7-based system) can further ensure optimal expression of the enzyme. The use of a standard bacterial (non-viral/non-phage) promoter may in some cases be detrimental to expression, as these promoters are often down-regulated during phage infection (in order for the phage to prioritize the bacterial resources for phage protein production). Thus, in some embodiments, the phage is preferably engineered to encode and express at high levels an indicator protein product.

In some embodiments, a recombinant indicator phage is constructed from a bacteriophage specific for bacterial species capable of biofilm formation. Bacterial cells detectable by the present disclosure include, but are not limited to, all species of Staphylococcus, including, but not limited to S. aureus, Salmonella spp., Pseudomonas spp., Streptococcus spp., all strains of Escherichia coli, Listeria, including, but not limited to L. monocytogenes, Campylobacter spp., Bacillus spp., Bordetella pertussis, Campylobacter jejuni, Chlamydia pneumoniae, Clostridium perfringens, Enterobacter spp., Klebsiella pneumoniae, Mycoplasma pneumoniae, Salmonella typhi, Shigella sonnei, and Streptococcus spp.

Additional microorganisms the antibiotic resistance of which can be detected using the claimed methods and systems can be selected from the group consisting of Abiotrophia adiacens, Acinetobacter baumanii, Actinomycetaceae, Bacteroides, Cytophaga and Flexibacter phylum, Bacteroides fragilis, Bordetella pertussis, Bordetella spp., Campylobacter jejuni and E. coli, Candida albicans, Candida dubliniensis, Candida glabrata, Candida guilliermondii, Candida krusei, Candida lusitaniae, Candida parapsilosis, Candida tropicalis, Candida zeylanoides, Candida spp., Chlamydia pneumoniae, Chlamydia trachomatis, Clostridium spp., Corynebacterium spp., Cronobacter spp, Crypococcus neoformans, Cryptococcus spp., Cryptosporidium parvum, Entamoeba spp., Enterobacteriaceae group, Enterococcus casseliflavus-flavescens-gallinarum group, Enterococcus faecalis, Enterococcus faecium, Enterococcus gallinarum, Enterococcus spp., Escherichia coli and Shigella spp. group, Gemella spp., Giardia spp., Haemophilus influenzae, Klebsiella oxytoca, Klebsiella pneumoniae, Legionella pneumophila, Legionella spp., Leishmania spp., Mycobacteriaceae family, Mycoplasma pneumoniae, Neisseria gonorrhoeae, Pseudomonas aeruginosa, Pseudomonads group, Staphylococcus aureus, Staphylococcus epidermidis, Staphylococcus haemolyticus, Staphylococcus hominis, Staphylococcus saprophyticus, Staphylococcus spp., Streptococcus agalactiae, Streptococcus pneumoniae, Streptococcus pyogenes, and Streptococcus spp.

In certain embodiments, an indicator bacteriophage is derived from a Staphylococcus aureus, Staphylococcus epidermis, Enterococcus faecalis, Streptococcus viridans, Escherichia coli, Klebsiella pneumonia, Proteus mirabilis, or Pseudomonas aeruginosa-specific phage. In some embodiments, the indicator phage is derived from a bacteriophage that is highly specific for a particular pathogenic microorganism of interest.

As discussed herein, such phage may replicate inside of the bacteria to generate hundreds of progeny phage. Detection of the indicator gene inserted into the phage can be used as a measure of the bacteria in the sample. S. aureus phages include, but are not limited to phage K, SA1, SA2, SA3, SA11, SA77, SA 187, Twort, NCTC9857, Ph5, Ph9, Ph10, Ph12, Ph13, U4, U14, U16, and U46. Well-studied phages of E. coli include T1, T2, T3, T4, T5, T7, and lambda; other E. coli phages available in the ATCC collection, for example, include phiX174, S13, Ox6, MS2, phiV1, fd, PR772, and ZIK1. Alternatively, natural phage may be isolated from a variety of environmental sources. A source for phage isolation may be selected based on the location where a microorganism of interest is expected to be found.

As described above for the compositions of the invention, the phage is derived from T7, T4, T4-like, phage K, MP131, MP115, MP112, MP506, MP87, ISP, or another naturally occurring phage having a genome with at least 99, 98, 97, 96, 95, 94, 93, 92, 91 90, 89, 88, 87, 86, 85, 84, 83, 82, 81, 80, 79, 78, 77, 76, 75, 74, 73, 72, 71, or 70% homology to phages disclosed above. In some aspects, the invention comprises a recombinant phage comprising an indicator gene inserted into a late gene region of the phage. In some embodiments, the phage is in the genus Tequatrovirus or Kayvirus. In one embodiment, the recombinant phage is derived from phage K, ISP, or MP115. In certain embodiments, the recombinant phage is highly specific for a particular bacterium. For example, in certain embodiments, the recombinant phage is highly specific for MRSA. In an embodiment, the recombinant phage can distinguish MRSA from at least 100 other types of bacteria.

In some embodiments, the selected wild-type bacteriophage is from the Caudovirales order of phages. Caudovirales are an order of tailed bacteriophages with double-stranded DNA (dsDNA) genomes. Each virion of the Caudovirales order has an icosahedral head that contains the viral genome and a flexible tail. The Caudovirales order comprises five bacteriophage families: Myoviridae (long contractile tails), Siphoviridae (long non-contractile tails), Podoviridae (short non-contractile tails), Ackermannviridae, and Herelleviridae. The term myovirus can be used to describe any bacteriophage with an icosahedral head and a long contractile tail, which encompasses bacteriophages within both the Myoviridae and Herelleviridae families.

Moreover, phage genes thought to be nonessential may have unrecognized function. For example, an apparently nonessential gene may have an important function in elevating burst size such as subtle cutting, fitting, or trimming functions in assembly. Therefore, deleting genes to insert an indicator may be detrimental. Most phages can package DNA that is a few percent larger than their natural genome. With this consideration, a smaller indicator gene may be a more appropriate choice for modifying a bacteriophage, especially one with a smaller genome. OpLuc and NANOLUC® proteins are only about 20 kDa (approximately 500-600 bp to encode), while FLuc is about 62 kDa (approximately 1,700 bp to encode). Moreover, the reporter gene should not be expressed endogenously by the bacteria (i.e., is not part of the bacterial genome), should generate a high signal to background ratio, and should be readily detectable in a timely manner. Promega's NANOLUC® is a modified Oplophorus gracilirostris (deep sea shrimp) luciferase. In some embodiments, NANOLUC® combined with Promega's NANO-GLO®, an imidazopyrazinone substrate (furimazine), can provide a robust signal with low background.

In some indicator phage embodiments, the indicator gene can be inserted into an untranslated region to avoid disruption of functional genes, leaving wild-type phage genes intact, which may lead to greater fitness when infecting non-laboratory strains of bacteria. Additionally, including stop codons in all three reading frames may help to increase expression by reducing read-through, also known as leaky expression. This strategy may also eliminate the possibility of a fusion protein being made at low levels, which would manifest as background signal (e.g., luciferase) that cannot be separated from the phage.

An indicator gene may express a variety of biomolecules. The indicator gene is a gene that expresses a detectable product or an enzyme that produces a detectable product. For example, in one embodiment the indicator gene encodes a luciferase enzyme. Various types of luciferase may be used. In alternate embodiments, and as described in more detail herein, the luciferase is one of Oplophorus luciferase, Firefly luciferase, Lucia luciferase, Renilla luciferase, or an engineered luciferase. In some embodiments, the luciferase gene is derived from Oplophorus. In some embodiments, the indicator gene is a genetically modified luciferase gene, such as NANOLUC®.

Thus, in some embodiments, the present invention comprises a genetically modified bacteriophage comprising a non-bacteriophage indicator gene in the late (class III) gene region. In some embodiments, the non-native indicator gene is under the control of a late promoter. Using a viral late gene promoter ensures the reporter gene (e.g., luciferase) is not only expressed at high levels, like viral capsid proteins, but also does not shut down like endogenous bacterial genes or even early viral genes.

Genetic modifications to infectious agents may include insertions, deletions, or substitutions of a small fragment of nucleic acid, a substantial part of a gene, or an entire gene. In some embodiments, inserted or substituted nucleic acids comprise non-native sequences. A non-native indicator gene may be inserted into a bacteriophage genome such that it is under the control of a bacteriophage promoter. Thus, in some embodiments, the non-native indicator gene is not part of a fusion protein. That is, in some embodiments, a genetic modification may be configured such that the indicator protein product does not comprise polypeptides of the wild-type bacteriophage. In some embodiments, the indicator protein product is soluble. In some embodiments, the invention comprises a method for detecting a bacterium of interest comprising the step of incubating a test sample with such a recombinant bacteriophage.

In some embodiments, expression of the indicator gene in progeny bacteriophage following infection of host bacteria results in a free, soluble protein product. In some embodiments, the non-native indicator gene is not contiguous with a gene encoding a structural phage protein and therefore does not yield a fusion protein. Unlike systems that employ a fusion of an indicator protein product to the capsid protein (i.e., a fusion protein), some embodiments of the present invention express a soluble indicator or reporter (e.g., soluble luciferase). In some embodiments, the indicator or reporter is ideally free of the bacteriophage structure. That is, the indicator or reporter is not attached to the phage structure. As such, the gene for the indicator or reporter is not fused with other genes in the recombinant phage genome. This may greatly increase the sensitivity of the assay (down to a single bacterium), and simplify the assay, allowing the assay to be completed in two hours or less for some embodiments, as opposed to several hours due to additional purification steps required with constructs that produce detectable fusion proteins. Further, fusion proteins may be less active than soluble proteins due, e.g., to protein folding constraints that may alter the conformation of the enzyme active site or access to the substrate. If the concentration is 1,000 bacterial cells/mL of sample, for example, less than four hours may be sufficient for the assay.

Moreover, fusion proteins by definition limit the number of the moieties attached to subunits of a protein in the bacteriophage. For example, using a commercially available system designed to serve as a platform for a fusion protein would result in about 415 copies of the fusion moiety, corresponding to the about 415 copies of the gene 10B capsid protein in each T7 bacteriophage particle. Without this constraint, infected bacteria can be expected to express many more copies of the indicator protein product (e.g., luciferase) than can fit on the bacteriophage. Additionally, large fusion proteins, such as a capsid-luciferase fusion, may inhibit assembly of the bacteriophage particle, thus yielding fewer bacteriophage progeny. Thus, a soluble, non-fusion indicator gene product may be preferable.

In some embodiments, the indicator phage encodes a reporter, such as a detectable enzyme. The indicator gene product may generate light and/or may be detectable by a color change. Various appropriate enzymes are commercially available, such as alkaline phosphatase (AP), horseradish peroxidase (HRP), or luciferase (Luc). In some embodiments, these enzymes may serve as the indicator protein product. In some embodiments, Firefly luciferase is the indicator protein product. In some embodiments, Oplophorus luciferase is the indicator moiety. In some embodiments, NANOLUC® is the indicator protein product. Other engineered luciferases or other enzymes that generate detectable signals may also be appropriate indicator moieties.

In some embodiments, the use of a soluble indicator protein product eliminates the need to remove contaminating stock phage from the lysate of the infected sample cells. With a fusion protein system, any bacteriophage used to infect sample cells would have the indicator protein product attached, and would be indistinguishable from the daughter bacteriophage also containing the indicator protein product. As detection of sample bacteria relies on the detection of a newly created (de novo synthesized) indicator protein product, using fusion constructs requires additional steps to separate old (stock phage) indicator from newly synthesized indicator. This may be accomplished by washing the infected cells multiple times, prior to the completion of the bacteriophage life cycle, inactivating excess stock phage after infection by physical or chemical means, and/or chemically modifying the stock bacteriophage with a binding moiety (such as biotin), which can then be bound and separated (such as by Streptavidin-coated Sepharose beads). However, even with all these attempts at removal, stock phage can remain when a high concentration of stock phage is used to assure infection of a low number of sample cells, creating background signal that may obscure detection of signal from infected cell progeny phage.

By contrast, with the soluble indicator protein product expressed in some embodiments of the present invention, purification of the stock phage from the final lysate is unnecessary, as the stock phage compositions do not have any indicator protein product. Thus, any indicator protein product present after infection must have been created de novo, indicating the presence of an infected bacterium or bacteria. To take advantage of this benefit, the production and preparation of phage may include purification of the phage from any free indicator protein product produced during the production of recombinant bacteriophage in bacterial culture. Standard bacteriophage purification techniques may be employed to purify some embodiments of phage according to the present invention, such as sucrose density gradient centrifugation, cesium chloride isopycnic density gradient centrifugation, HPLC, size exclusion chromatography, and dialysis or derived technologies (such as Amicon brand concentrators—Millipore, Inc.). Cesium chloride isopycnic ultracentrifugation can be employed as part of the preparation of recombinant phage of the disclosure, to separate stock phage particles from contaminating luciferase protein produced upon propagation of the phage in the bacterial host. In this way, the recombinant bacteriophages of the invention are substantially free of any luciferase generated during production in the bacteria. Removal of residual luciferase present in the phage stock can substantially reduce background signal observed when the recombinant bacteriophages are incubated with a test sample.

In some embodiments of the modified recombinant bacteriophage, the late promoter (class III promoter) has high affinity for RNA polymerase of the same bacteriophage that transcribes genes for structural proteins assembled into the bacteriophage particle. These proteins are the most abundant proteins made by the phage, as each bacteriophage particle comprises dozens or hundreds of copies of these molecules. The use of a viral late promoter can ensure optimally high level of expression of the luciferase indicator protein product. The use of a late viral promoter derived from, specific to, or active under the original wild-type bacteriophage the indicator phage is derived from can further ensure optimal expression of the indicator protein product. For example, indicator phage specific for MRSA may comprise the consensus late gene promoter from S. aureus phage ISP. The use of a standard bacterial (non-viral/non-bacteriophage) promoter may in some cases be detrimental to expression, as these promoters are often down-regulated during bacteriophage infection (in order for the bacteriophage to prioritize the bacterial resources for phage protein production). Thus, in some embodiments, the phage is preferably engineered to encode and express at high level a soluble (free) indicator moiety, using a placement in the genome that does not limit expression to the number of subunits of a phage structural component.

Compositions of the disclosure may comprise one or more wild-type or genetically modified infectious agents (e.g., bacteriophages) and one or more indicator genes. In some embodiments, compositions can include cocktails of different indicator phages that may encode and express the same or different indicator proteins. In some embodiments, the cocktail of indicator bacteriophages comprises at least two different types of recombinant bacteriophages.

### Therapeutically-Effective Bacteriophages

As described in more detail herein, the compositions, methods, and systems of the present disclosure may comprise infectious agents for use in the diagnosis and treatment of biofilm-related infections. In certain embodiments, the disclosure comprises a therapeutically-effective bacteriophage. In some embodiments, the therapeutically-effective bacteriophage is a wild-type bacteriophage. In other embodiments, the therapeutically-effective bacteriophage is a recombinant bacteriophage, wherein the bacteriophage genome is genetically modified to include a genetic construct comprising a gene encoding an enzyme.

In certain embodiments, the gene does not encode a fusion protein. For example, in certain embodiments, expression of the enzyme during bacteriophage replication following infection of a host bacterium results in production of a free enzyme. In some instances, the genetic construct may further comprise an exogenous promoter. In certain embodiments, the genetic construct may be inserted into a late gene region of the bacteriophage. Late genes are generally expressed at higher levels than other phage genes, as they code for structural proteins. The late gene region may be a class III gene region and may include a gene for a major capsid protein.

In some embodiments of modified phage, the additional, exogenous late promoter (class III promoter, e.g., from phage K or T7 or T4) has high affinity for RNA polymerase of the same native phage (e.g., phage K or T7 or T4, respectively) that transcribes genes for structural proteins assembled into the phage particle. These proteins are the most abundant proteins made by the phage, as each phage particle comprises dozens or hundreds of copies of these molecules. The use of a viral late promoter can ensure optimally high level of expression of the enzyme. The use of a late viral promoter derived from, specific to, or active under the original wild-type phage the therapeutic phage is derived from (e.g., the phage K or T4 or T7 late promoter with a phage K- or T4- or T7-based system) can further ensure optimal expression of the enzyme. The use of a standard bacterial (non-viral/non-phage) promoter may in some cases be detrimental to expression, as these promoters are often down-regulated during phage infection (in order for the phage to prioritize the bacterial resources for phage protein production). Thus, in some embodiments, the phage is preferably engineered to encode and express at high levels an enzyme.

Biofilms are an aggregation of bacterial cells surrounded by an extracellular matrix, which allows the bacteria to adhere to inert (e.g., implanted medical devices) or living surfaces. Additionally, biofilms increase the chance of infection in a subject and have been shown to be resistant to both antibiotics and phagocytes. Bacteriophages are known to produce enzymes capable of breaking down extracellular matrix, and thus, are able to target bacteria within biofilms.

Bacteriophages are known to naturally produce enzymes capable of breaking down the biofilm matrix. In some instances, bacteriophage genomes contain genes encoding soluble enzymes that are intended to penetrate the cell wall. These enzymes are capable of hydrolyzing the cell wall of bacteria, and thus, allow the phages to escape the cell. The composition of the extracellular matrix surrounding biofilms is similar to that of a bacterial cell wall, thus, increasing expression of bacteriophage enzymes can be advantageous for the treatment of biofilm-related infections. In some embodiments, bacteriophages are modified to increase the level of enzyme (e.g., lysin and endolysin) produced or to allow for the production of a different enzyme. Additionally, some bacteriophages (e.g., T4) have additional enzymes present on the bacteriophage tail that further aid in the penetration of bacterial cell walls. However, during the natural infection process, these enzymes are masked until the tail reconfigures during the infection cycle.

In some embodiments, the bacteriophage is modified to allow for production of an enzyme specific for a microorganism of interest. In some embodiments the bacteriophage is genetically engineered to include virulence-enhancing factors. In some embodiments, a gene encoding an enzyme is inserted into the bacteriophage genome. Upon infection of bacterial cells, the inserted gene encoding an enzyme is produced at high levels and is released into the extracellular matrix of the biofilm from lysed bacterial cells. In certain embodiments, the enzyme is a glycosidase, amidase, or endopeptidase. Glycosidase, amidases, and endopeptidases are the main enzymes produced by phages for the lysis of the cell or injection of DNA through the cell wall. For example, in some embodiments, a therapeutic recombinant bacteriophage may be specific for Staphylococcus infections. Staphylococcus-specific phage containing dispersin B (DspB), a glycoside hydrolase enzyme that is produced by Actinobacillus actinomycetemcomitans and hydrolyzes β-1,6-N-acetyl-D-glucosamine may be used to treat biofilm-related Staphylococcus infections.

In other embodiments, the bacteriophage is modified to enhance production of a naturally occurring enzyme. For example, such an enzyme may be inserted into the phage genome recombinantly, either creating a fusion protein on the virion surface or as a soluble protein that may diffuse into the biofilm from infected bacteria. This may be done through homologous recombination cloning, CRISPR based cloning, or by any other method generally known in the art.

### Methods of Using Bacteriophages for the Diagnosis and Treatment of Biofilm-Related Infections

As noted herein, in certain embodiments, the invention may comprise methods of using infectious particles for detecting microorganisms. The methods of the invention may be embodied in a variety of ways.

In some embodiments, the diagnostic recombinant bacteriophage are capable of determining the bacterial strain(s) present in a biofilm-related infection. Detection of the bacterial strain(s) present in the biofilm is important for determining the appropriate treatment of the infection.

In one embodiment, the invention may comprise a method for the diagnosis and treatment of a biofilm-related infection in a subject with an implant comprising the steps of: (i) providing a biological sample taken from the subject with an implant; (ii) diagnosing the subject with a biofilm-related infection by detecting the presence of at least one microorganism of interest comprising the steps of: (a) contacting at least one aliquot of the biological sample with an amount of a reporter cocktail composition comprising at least one recombinant bacteriophage; (b) detecting a signal produced following replication of the recombinant bacteriophage, wherein detection of the signal indicates the microorganism of interest is present in the sample; and (iii) treating the subject diagnosed with a biofilm-related infection comprising the steps of: (a) selecting a therapeutic cocktail composition based on the diagnosis of step (ii); administering a therapeutically-effective amount of a therapeutic cocktail composition comprising at least one bacteriophage, wherein the bacteriophage is specific for the detected microorganism of interest; and (c) optionally administering at least one additional therapeutic agent.

In certain embodiments, the step of diagnosing the subject with a biofilm-related infection comprises detecting at least one microorganism of interest. In an embodiment, the method for detecting at least one microorganism of interest in a sample comprises the steps of: incubating the sample with bacteriophage that infects the bacterium of interest, wherein the bacteriophage comprises a genetic construct, and wherein the genetic construct comprises an indicator gene such that expression of the indicator gene during bacteriophage replication following infection of the bacterium of interest results in production of a soluble indicator protein product; and detecting the indicator protein product, wherein positive detection of the indicator protein product indicates that the microorganism of interest is present in the sample. In certain embodiments, the genetic construct further comprises and additional exogenous promoter.

In some embodiments, the assay may be performed to utilize a general concept that can be modified to accommodate different sample types or sizes and assay formats. Embodiments employing recombinant bacteriophage of the invention (i.e., indicator bacteriophage) may allow rapid detection of specific bacterial strains with total assay times under 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12, 12.5, 13.0, 13.5, 14.0, 14.5, 15.0, 15.5, 16.0, 16.5, 17.0, 17.5, 18.0, 18.5, 19.0, 19.5, 20.0, 21.0, 21.5 22.0, 22.5, 23.0, 23.5, 24.0, 24.5 25.0, 25.5, or 26.0 hours, depending on the sample type, sample size, and assay format. For example, the amount of time required may be somewhat shorter or longer depending on the strain of bacteriophage and the strain of bacteria to be detected in the assay, type and size of the sample to be tested, conditions required for viability of the target, complexity of the physical/chemical environment, and the concentration of “endogenous” non-target bacterial contaminants. For example, detection for the presence of Gram-negative strains (e.g., E. coli, Klebsiella, Shigella) may be completed with total assay times under 0.5, 1.0, 1.5, 2.0, 2.5, or 3.0 hours without detecting for antibiotic resistance or total assay times under 1.5, 2.0, 2.5, 3.0, 3.5, 4.0, or 4.5 hours with detecting for antibiotic resistance. Detection for the presence of Gram-positive strains may be completed with total assay times under 1.0, 1.5, 2.0, 2.5, 3.0, 3.5, 4.0, or 4.5 hours without detecting antibiotic resistance or 2.0, 3.0, 4.0, 4.5, 5.0, 5.5, 6.0, or 6.5 hours with detecting antibiotic resistance.

The bacteriophage (e.g., Phage K, ISP, MP115) may be engineered to express a soluble luciferase during replication of the phage. Expression of luciferase is driven by a viral capsid promoter (e.g., the bacteriophage Pecentumvirus or T4 late promoter), yielding high expression. Stock phage are prepared such that they are free of luciferase, so the luciferase detected in the assay must come from replication of progeny phage during infection of the bacterial cells. Thus, there is generally no need to separate out the parental phage from the progeny phage.

In some embodiments, enrichment of bacteria in the sample is not needed prior to testing. In some embodiments, the sample may be enriched prior to testing by incubation in conditions that encourage growth. In such embodiments, the enrichment period can be 1, 2, 3, 4, 5, 6, 7, 8, 9, 10, 11, 12, 13, 14, 15, 16, 17, 18, 19, 20, 21, 22, 23, or 24 hours or longer, depending on the sample type and size.

In some embodiments, the indicator bacteriophage comprises a detectable indicator protein product, and infection of a single pathogenic cell (e.g., bacterium) can be detected by an amplified signal generated via the indicator protein product. Thus, the method may comprise detecting an indicator protein product produced during phage replication, wherein detection of the indicator indicates that the bacterium of interest is present in the sample.

In an embodiment, the invention may comprise a method for detecting a bacterium of interest in a sample comprising the steps of: incubating the sample with a recombinant bacteriophage that infects the bacterium of interest, wherein the recombinant bacteriophage comprises an indicator gene inserted into a late gene region of the bacteriophage such that expression of the indicator gene during bacteriophage replication following infection of host bacteria results in production of a soluble indicator protein product; and detecting the indicator protein product, wherein positive detection of the indicator protein product indicates that the bacterium of interest is present in the sample. In some embodiments, the amount of indicator protein product detected corresponds to the amount of the bacterium of interest present in the sample. In some embodiments, positive detection of a particular bacterium of interest is used to diagnose the subject with a biofilm-related infection.

As described in more detail herein, the compositions, methods, and systems of the disclosure may utilize a range of concentrations of parental indicator bacteriophage to infect bacteria present in the sample. In some embodiments the indicator bacteriophage are added to the sample at a concentration sufficient to rapidly find, bind, and infect target bacteria that are present in very low numbers in the sample, such as ten cells. In some embodiments, the phage concentration can be sufficient to find, bind, and infect the target bacteria in less than one hour. In other embodiments, these events can occur in less than two hours, or less than three hours, or less than four hours, following addition of indicator phage to the sample. For example, in certain embodiments, the bacteriophage concentration for the incubating step is greater than 1×10⁵ PFU/mL, greater than 1×10⁶ PFU/mL, or greater than 1×10⁷ PFU/mL, or greater than 1×10⁸ PFU/mL.

In certain embodiments, the recombinant stock phage composition may be purified so as to be free of any residual indicator protein that may be generated upon production of the phage stock. Thus, in certain embodiments, the recombinant bacteriophage may be purified using a sucrose gradient or cesium chloride isopycnic density gradient centrifugation prior to incubation with the sample. When the infectious agent is a bacteriophage, this purification may have the added benefit of removing bacteriophage that do not have DNA (i.e., empty phage or “ghosts”).

In some embodiments of the methods of the invention, the microorganism may be detected without any isolation or purification of the microorganisms from a sample. For example, in certain embodiments, a sample containing one or a few microorganisms of interest may be applied directly to an assay container such as a spin column, a microtiter well, or a filter and the assay is conducted in that assay container. Various embodiments of such assays are disclosed herein.

In some embodiments, at least one aliquot of a biological sample is contacted with an amount of an indicator bacteriophage cocktail composition. In certain instances, the indicator cocktail composition comprises at least one recombinant bacteriophage specific for a particular bacterium of interest. In other embodiments, the indicator cocktail composition comprises at least two, at least three, at least four, at least five, at least six, at least seven, at least eight, at least nine, or at least ten types of recombinant bacteriophages specific for a particular bacterium of interest. In certain embodiments, the step of diagnosing the subject with a biofilm-related infection further comprises contacting a plurality of aliquots of the biological samples with a plurality of indicator cocktail compositions. In some instances, each indicator cocktail composition is specific for a different microorganism of interest. For example, a first aliquot may be contacted with a recombinant bacteriophage cocktail composition specific for Enterococcus faecalis, a second aliquot may be contacted with a recombinant bacteriophage cocktail composition specific for Staphylococcus aureus, a third aliquot may be contacted with a recombinant bacteriophage cocktail composition specific for Staphylococcus epidermidis, a fourth aliquot may be contacted with a recombinant bacteriophage cocktail composition specific for Streptococcus viridans, a fifth aliquot may be contacted with a recombinant bacteriophage cocktail composition specific for Escherichia coli, a sixth aliquot may be contacted with a recombinant bacteriophage cocktail composition specific for Klebsiella pneumoniae, a seventh aliquot may be contacted with a recombinant bacteriophage cocktail composition specific for Proteus mirabilis, and an eighth aliquot may be contacted with a recombinant bacteriophage cocktail composition specific for Pseudomonas aeruginosa. In some embodiments, at least 1, 2, 3, 4, 5, 6, 7, 8, 9, 10, 11, 12, 13, 14, 15, 16, 17, 18, 19, 20, 21, 22, 23, 24, or 25 aliquots of the biological sample are contacted with at least 1, 2, 3, 4, 5, 6, 7, 8, 9, 10, 11, 12, 13, 14, 15, 16, 17, 18, 19, 20, 21, 22, 23, 24, or 25 different reporter cocktail compositions.

Aliquots of a test sample may be distributed directly into wells of a multi-well plate, indicator phage may be added, and after a period of time sufficient for infection, a lysis buffer may be added as well as a substrate for the indicator moiety (e.g., luciferase substrate for a luciferase indicator) and assayed for detection of the indicator signal. Some embodiments of the method can be performed on filter plates or 96 well plates. Some embodiments of the method can be performed with or without concentration of the sample before infection with indicator phage.

For example, in many embodiments, multi-well plates are used to conduct the assays. The choice of plates (or any other container in which detecting may be performed) may affect the detecting step. For example, some plates may include a colored or white background, which may affect the detection of light emissions. Generally speaking, white plates have higher sensitivity but also yield a higher background signal. Other colors of plates may generate lower background signal but also have a slightly lower sensitivity. Additionally, one reason for background signal is the leakage of light from one well to another, adjacent well. There are some plates that have white wells but the rest of the plate is black. This allows for a high signal inside the well but prevents well-to-well light leakage and thus may decrease background. Thus the choice of plate or other assay vessel may influence the sensitivity and background signal for the assay.

Methods of the disclosure may comprise various other steps to increase sensitivity. For example, as discussed in more detail herein, the method may comprise a step for washing the captured and infected bacterium, after adding the bacteriophage but before incubating, to remove excess bacteriophage and/or luciferase or other reporter protein contaminating the bacteriophage preparation.

In some embodiments, detection of the microorganism of interest may be completed without the need for culturing the sample as a way to increase the population of the microorganisms. For example, in certain embodiments the total time required for detection is less than 28.0 hours, 27.0 hours, 26.0 hours, 25.0 hours, 24.0 hours, 23.0 hours, 22.0 hours, 21.0 hours, 20.0 hours, 19.0 hours, 18.0 hours, 17.0 hours, 16.0 hours, 15.0 hours, 14.0 hours, 13.0 hours, 12.0 hours, 11.0 hours, 10.0 hours, 9.0 hours, 8.0 hours, 7.0 hours, 6.0 hours, 5.0 hours, 4.0 hours, 3.0 hours, 2.5 hours, 2.0 hours, 1.5 hours, or less than 1.0 hour. Minimizing time to result is critical in diagnostic testing.

In contrast to assays known in the art, the method of the disclosure can detect individual microorganisms. Thus, in certain embodiments, the method may detect as few as 10 cells of the microorganism present in a sample. For example, in certain embodiments, the recombinant indicator bacteriophage is highly specific for Staphylococcus spp., E. coli strains, Shigella spp., Klebsiella spp., or Pseudomonas spp. In an embodiment, the recombinant indicator bacteriophage can distinguish a bacterium of interest in the presence of other types of bacteria. In certain embodiments, the recombinant bacteriophage can be used to detect a single bacterium of the specific type in the sample. In certain embodiments, the recombinant indicator bacteriophage detects as few as 2, 3, 4, 5, 6, 7, 8, 9, 10, 15, 20, 30, 40, 50, 60, 70, 80, 90, or 100 of the specific bacteria in the sample.

Thus, aspects of the present disclosure provide methods for detection of microorganisms in a test sample via an indicator protein product. In some embodiments, where the microorganism of interest is a bacterium, the indicator protein product may be associated with an infectious agent such as an indicator bacteriophage. The indicator protein product may react with a substrate to emit a detectable signal or may emit an intrinsic signal (e.g., bioluminescent protein). In some embodiments, the detection sensitivity can reveal the presence of as few as 50, 20, 10, 9, 8, 7, 6, 5, 4, 3, or 2 cells of the microorganism of interest in a test sample. In some embodiments, even a single cell of the microorganism of interest may yield a detectable signal. In some embodiments, the bacteriophage is a Phage K, ISP, or MP115. In certain embodiments, a recombinant Staphylococcus spp.-specific bacteriophage is highly specific for Staphylococcus spp.

In some embodiments, the indicator protein product encoded by the recombinant indicator bacteriophage may be detectable during or after replication of the bacteriophage. Many different types of detectable biomolecules suitable for use as indicator moieties are known in the art, and many are commercially available. In some embodiments the indicator phage comprises an enzyme, which serves as the indicator moiety. In some embodiments, the genome of the indicator phage is modified to encode a soluble protein. In some embodiments, the indicator phage encodes a detectable enzyme. The indicator may emit light and/or may be detectable by a color change in an added substrate. Various appropriate enzymes are commercially available, such as alkaline phosphatase (AP), horseradish peroxidase (HRP), or luciferase (Luc). In some embodiments, these enzymes may serve as the indicator moiety. In some embodiments, Firefly luciferase is the indicator moiety. In some embodiments, Oplophorus luciferase is the indicator moiety. In some embodiments, NANOLUC® is the indicator moiety. Other engineered luciferases or other enzymes that generate detectable signals may also be appropriate indicator moieties.

Thus, in some embodiments, the recombinant indicator bacteriophage of the compositions, methods, or systems is prepared from wild-type bacteriophage. In some embodiments, the indicator gene encodes a protein that emits an intrinsic signal, such as a fluorescent protein (e.g., green fluorescent protein or others). The indicator may emit light and/or may be detectable by a color change. In some embodiments, the indicator gene encodes an enzyme (e.g., luciferase) that interacts with a substrate to generate signal. In some embodiments, the indicator gene is a luciferase gene. In some embodiments, the luciferase gene is one of Oplophorus luciferase, Firefly luciferase, Renilla luciferase, External Gaussia luciferase, Lucia luciferase, or an engineered luciferase such as NANOLUC®, Rluc8.6-535, or Orange Nano-lantern.

Detecting the indicator may include detecting emissions of light. In some embodiments, the indicator protein product (e.g., luciferase) is reacted with a substrate to produce a detectable signal. The detection of the signal can be achieved with any machine or device generally known in the art. In some embodiments, the signal can be detected using an In Vivo Imaging System (IVIS). The IVIS uses a CCD camera or a CMOS sensor to measure light emissions by total flux. Total flux=radiance (photons/second). Average radiance is measured as photons/second/cm²/steradian. In other embodiments, the detection of the signal can be achieved with a luminometer, a spectrophotometer, CCD camera, or CMOS camera may detect color changes and other light emissions. In some embodiments the signal is measured as absolute RLU. In further embodiments, the signal to background ratio needs to be high (e.g., >2.0, >2.5, or >3.0) in order for single cells or low numbers of cells to be detected reliably.

In some embodiments, the indicator phage is genetically engineered to contain the gene for an enzyme, such as a luciferase, which is only produced upon infection of bacteria that the phage specifically recognizes and infects. In some embodiments, the indicator moiety is expressed late in the viral life cycle. In some embodiments, as described herein, the indicator is a soluble protein (e.g., soluble luciferase) and is not fused with a phage structural protein that limits its copy number.

Thus in some embodiments utilizing indicator phage, the invention comprises a method for detecting a microorganism of interest comprising the steps of capturing at least one sample bacterium; incubating the at least one bacterium with a plurality of indicator phage; allowing time for infection and replication to generate progeny phage and express soluble indicator moiety; and detecting the progeny phage, or preferably the indicator, wherein detection of the indicator demonstrates that the bacterium is present in the sample.

For example, in some embodiments the test sample bacterium may be captured by binding to the surface of a plate, or by filtering the sample through a bacteriological filter (e.g., 0.45 μm pore size spin filter or plate filter). In an embodiment, the infectious agent (e.g., indicator phage) is added in a minimal volume to the captured sample directly on the filter. In an embodiment, the microorganism captured on the filter or plate surface is subsequently washed one or more times to remove excess unbound infectious agent. In an embodiment, a medium (e.g., Luria-Bertani (LB) Broth, Buffered Peptone Water (BPW) or Tryptic Soy Broth or Tryptone Soy Broth (TSB), Brain Heart Infusion (BHI) may be added for further incubation time, to allow replication of bacterial cells and phage and high-level expression of the gene encoding the indicator moiety. However, a surprising aspect of some embodiments of testing assays is that the incubation step with indicator phage only needs to be long enough for a single phage life cycle. A single replication cycle of indicator phage can be sufficient to facilitate sensitive and rapid detection according to some embodiments of the present invention.

In some embodiments, aliquots of a test sample comprising bacteria may be applied to a spin column and after infection with a recombinant bacteriophage and an optional washing to remove any excess bacteriophage, the amount of soluble indicator detected will be proportional to the amount of bacteriophage that are produced by infected bacteria.

Soluble indicator (e.g., luciferase) released into the surrounding liquid upon lysis of the bacteria may then be measured and quantified. In an embodiment, the solution is spun through the filter, and the filtrate collected for assay in a new receptacle (e.g., in a luminometer) following addition of a substrate for the indicator enzyme (e.g., luciferase substrate).

In various embodiments, the purified parental indicator phage does not comprise the detectable indicator itself, because the parental phage can be purified before it is used for incubation with a test sample. Expression of late (class III) genes occurs late in the viral life cycle. In some embodiments of the present invention, parental phage may be purified to exclude any existing indicator protein (e.g., luciferase). In some embodiments, expression of the indicator gene during bacteriophage replication following infection of host bacteria results in a soluble indicator protein product. Thus, in many embodiments, it is not necessary to separate parental from progeny phage prior to the detecting step. In an embodiment, the microorganism is a bacterium and the indicator phage is a bacteriophage. In an embodiment, the indicator protein product is a free, soluble luciferase, which is released upon lysis of the host microorganism.

The assay may be performed in a variety of ways. In one embodiment, the sample is added to at least one well on a 96-well plate, incubated with phage, lysed, incubated with substrate, and then read. In other embodiments, the sample is added to a 96-well filter plate, the plate is centrifuged and media is added to bacteria collected on the filter before being incubated with phage. In still other embodiments, the sample is captured on at least one well of a 96-well plate using antibodies and washed with media to remove excess cells before being incubated with phage.

In some embodiments, lysis of the bacterium may occur before or during the detection step. Experiments suggest that infected unlysed cells may be detectable upon addition of luciferase substrate in some embodiments. Presumably, luciferase may exit cells and/or luciferase substrate may enter cells without complete cell lysis. For example, in some embodiments the substrate for the luciferase is cell-permeable (e.g., furimazine). Thus, for embodiments utilizing the spin filter system, where only luciferase released into the lysate (and not luciferase still inside intact bacteria) is analyzed in the luminometer, lysis is required for detection. However, for embodiments utilizing filter plates or 96-well plates with sample in solution or suspension, where the original plate full of intact and lysed cells is directly assayed in the luminometer, lysis is not necessary for detection.

In some embodiments, the reaction of indicator moiety (e.g., luciferase) with substrate may continue for 60 minutes or more, and detection at various time points may be desirable for optimizing sensitivity. For example, in embodiments using 96-well filter plates as the solid support and luciferase as the indicator, luminometer readings may be taken initially and at 10- or 15-minute intervals until the reaction is completed.

Surprisingly, high concentrations of phage utilized for infecting test samples have successfully achieved detection of very low numbers of a target microorganism in a very short timeframe. The incubation of phage with a test sample in some embodiments need only be long enough for a single phage life cycle. In some embodiments, the bacteriophage concentration for this incubating step is greater than 1.0×10⁶, 2.0×10⁶, 3.0 10⁶, 5.0×10⁶, 6.0×10⁶, 7.0×10⁶, 8.0×10⁶, 9.0×10⁶, 1.0×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2.0×10⁷, 3.0×10⁷, 4.0×10⁷, 5.0×10⁷, 6.0×10⁷, 7.0×10⁷, 8.0×10⁷, 9.0×10⁷, or 1.0×10⁸ PFU/mL.

Success with such high concentrations of phage is surprising because the large numbers of phage were previously associated with “lysis from without,” which killed target cells and thereby prevented generation of useful signal from earlier phage assays. It is possible that the clean-up of prepared phage stocks described herein helps to alleviate this problem (e.g., clean-up by sucrose gradient or cesium chloride isopycnic density gradient ultracentrifugation), because in addition to removing any contaminating luciferase associated with the phage, this clean-up may also remove ghost particles (particles that have lost DNA). The ghost particles can lyse bacterial cells via “lysis from without,” killing the cells prematurely and thereby preventing generation of indicator signal. Electron microscopy demonstrates that a crude phage lysate (i.e., before cesium chloride clean-up) may have greater than 50% ghosts. These ghost particles may contribute to premature death of the microorganism through the action of many phage particles puncturing the cell membrane. Thus ghost particles may have contributed to previous problems where high PFU concentrations were reported to be detrimental. Moreover, a very clean phage prep allows the assay to be performed with no wash steps, which makes the assay possible to perform without an initial concentration step. Some embodiments do include an initial concentration step, and in some embodiments this concentration step allows a shorter enrichment incubation time.

Some embodiments of testing methods may further include confirmatory assays. A variety of assays are known in the art for confirming an initial result, usually at a later point in time. For example, the samples can be cultured (e.g., selective chromogenic plating), and PCR can be utilized to confirm the presence of the microbial DNA, or other confirmatory assays can be used to confirm the initial result.

In certain embodiments, the methods of the present disclosure combine the use of a binding agent (e.g., antibody) to purify and/or concentrate a microorganism of interest such as Staphylococcus spp. from the sample in addition to detection with an infectious agent. For example, in certain embodiments, the invention comprises a method for detecting a microorganism of interest in a sample comprising the steps of: capturing the microorganism from the sample on a prior support using a capture antibody specific to the microorganism of interest such as Staphylococcus spp.; incubating the sample with a recombinant bacteriophage that infects Staphylococcus spp. wherein the recombinant bacteriophage comprises an indicator gene inserted into a late gene region of the bacteriophage such that expression of the indicator gene during bacteriophage replication following infection of host bacteria results in a soluble indicator protein product; and detecting the indicator protein product, wherein positive detection of the indicator protein product indicates that Staphylococcus spp. is present in the sample.

In some embodiments, synthetic phage are designed to optimize desirable traits for use in pathogen detection assays. In some embodiments, bioinformatics and previous analyses of genetic modifications are employed to optimize desirable traits. For example, in some embodiments, the genes encoding phage tail proteins can be optimized to recognize and bind to particular species of bacteria. In other embodiments the genes encoding phage tail proteins can be optimized to recognize and bind to an entire genus of bacteria, or a particular group of species within a genus. In this way, the phage can be optimized to detect broader or narrower groups of pathogens. In some embodiments, the synthetic phage may be designed to improve expression of the indicator gene. Additionally and/or alternatively, in some instances, the synthetic phage may be designed to increase the burst size of the phage to improve detection.

In some embodiments, the stability of the phage may be optimized to improve shelf-life. For example, enzybiotic solubility may be increased in order to increase subsequent phage stability. Additionally and/or alternatively phage thermostability may be optimized. Thermostable phage better preserve functional activity during storage thereby increasing shelf-life. Thus, in some embodiments, the thermostability and/or pH tolerance may be optimized.

In some embodiments, the genetically modified phage or the synthetically derived phage comprises a detectable indicator. In some embodiments the indicator is a luciferase. In some embodiments the phage genome comprises an indicator gene (e.g., a luciferase gene or another gene encoding a detectable indicator).

In some embodiments, the detection recombinant bacteriophage are used to diagnose the presence of a biofilm-related infection and identify the specific strains responsible for the biofilm. The diagnosis can then be used to select an appropriate treatment.

In certain embodiments, a therapeutic cocktail composition is selected based off the determined diagnosis. For example, if Staphylococcus spp. are detected during the diagnosis of the subject, then a therapeutic cocktail composition comprising recombinant bacteriophage specific for Staphylococcus spp. will be selected. In other embodiments, a broad-spectrum therapeutic cocktail composition is selected to treat multiple potential infections.

In some embodiments, the therapeutic cocktail composition comprises at least one type of bacteriophage. In other embodiments, the therapeutic cocktail composition comprises at least 2, 3, 4, 5, 6, 7, 8, 9, or 10 types of bacteriophages. These bacteriophages may be specific for the same species of bacteria or for different species of bacteria. In some embodiments, the bacteriophages are wild-type bacteriophages. In other embodiments, the bacteriophages are recombinant bacteriophages.

Biofilm-related infections are difficult to treat due to the inability of typical antimicrobials (e.g., antibiotics) to break down the biofilm. As an alternative or complementation to antibiotic treatment, bacteriophage that have been genetically modified to express an enzyme that is capable of hydrolyzing the bacterial cell may be used. These phages are able to infect the bacterial cells of the biofilm, replicate to produce progeny phage, and also produce an enzyme that can break down the biofilm. As the infection progresses, the progeny bacteriophage continue to infect other bacterial cells, which then release the enzyme into the environment, thereby removing the biofilm.

In some embodiments, a therapeutically-effective amount of a therapeutic cocktail composition is administered to a subject diagnosed with a biofilm-related infection. In some embodiments, the therapeutic cocktail composition is administered intravenously (e.g., to treat prosthetic heart valve infections). In other embodiments, the therapeutic cocktail composition is administered locally (e.g., to treat prosthetic joint infections). In some embodiments, repeated dosages of the therapeutic cocktail composition are administered. The frequency of dosages may vary based on the severity of infection, specific phage, and route of administration. For example, the therapeutic cocktail composition may be administered every 2 hours, 4 hours, 8 hours, 12 hours, 24 hours, or 48 hours. The therapeutically-effective amount of the cocktail composition will also vary depending on which phage is used. In some embodiments the therapeutically-effective amount of the therapeutic cocktail composition will comprise at least one therapeutic phage with a concentration greater than 1.0×10⁶, 1.0×10⁷, 1.0×10⁸, 1.0×10⁹, 1.0×10¹⁰, 1.0×10¹¹, 1.0×10¹².

In additional embodiments, at least one additional therapeutic agent is administered. In some embodiments, the additional therapeutic agent is an antibiotic. Non-limiting examples of antibiotics that can be used in the invention include aminoglycosides, carbacephems, carbapenems, cephalosporins, glycopeptides, macrolides, monobactams, penicillin, beta-lactam antibiotic, quinolones, bacitracin, sulfonamides, tetracyclines, streptogramines, chloramphenicol, clindamycin, and lincosamide, cephamycins, lincomycins, daptomycin, oxazolidinone, and glycopeptide antibiotic.

In another aspect, the present disclosure comprises a method of preventing or inhibiting infection in a subject comprising applying a cocktail composition comprising at least one recombinant bacteriophage to a surgical implant, dressing, or suture. In certain instances, the cocktail composition comprises therapeutic recombinant bacteriophage. In further embodiments, a cocktail composition comprises at least 2, 3, 4, 5, 6, 7, 8, 9, 10, 11, 12, 13, 14, 15, 16, 17, 18, 19, 20, 21, 22, 23, 24, or 25 types of recombinant bacteriophage. The recombinant bacteriophage comprising the cocktail composition may be specific for the same or different species of bacteria.

In yet another aspect, the present disclosure comprises a surgical implant, dressing, or suture coated in a cocktail composition comprising at least one recombinant bacteriophage. In certain instances, the cocktail composition comprises therapeutic recombinant bacteriophage. In further embodiments, a cocktail composition comprises at least 2, 3, 4, 5, 6, 7, 8, 9, 10, 11, 12, 13, 14, 15, 16, 17, 18, 19, 20, 21, 22, 23, 24, or 25 types of recombinant bacteriophage. The recombinant bacteriophage comprising the cocktail composition may be specific for the same or different species of bacteria.

### Determining Antibiotic Resistance

In some aspects, the invention comprises a method for detecting antibiotic resistance of a microorganism. In some embodiments, the disclosure provides methods for detecting antibiotic-resistant microorganisms in a sample comprising: (a) contacting the sample with an antibiotic, (b) contacting the sample with an infectious agent, wherein the infectious agent comprises an indicator gene and is specific to the microorganism, and wherein the indicator gene encodes an indicator protein product, and (c) detecting a signal produced by an indicator protein product, wherein detection of the signal is used to determine antibiotic resistance.

The methods may use an infectious agent for detection of the microorganism of interest. For example, in certain embodiments, the microorganism of interest is a bacterium and the infectious agent is a phage. The antibiotic referred to in this application can be any agent that is bacteriostatic (capable of inhibiting the growth of a microorganism) or bactericidal (capable of killing a microorganism). Thus, in certain embodiments, the methods may comprise detection of resistance of a microorganism of interest in a sample to an antibiotic by contacting the sample with the antibiotic, and incubating the sample that has been contacted with antibiotic with an infectious agent that infects the microorganism of interest. This is distinct from those assays that detect the presence of genes (e.g., PCR) or proteins (e.g., antibody) that may confer antibiotic resistance, but do not test their functionality. Thus the current assay allows for phenotypic detection as opposed to genotypic detection.

In certain embodiments, the methods may comprise detection of a functional resistance gene in the microorganism of interest in a sample to an antibiotic. PCR allows for the detection of antibiotic-resistance genes; however, PCR is not able to distinguish between bacteria having functional antibiotic-resistance genes and those having non-functional antibiotic-resistance genes, thus, resulting in false-positive detection of antibiotic-resistant bacteria. The presently embodied methods, are capable of positively detecting bacteria with functional antibiotic-resistance genes, without positive detection of bacteria with non-functional antibiotic resistance genes. The method disclosed herein, allows detection of functional resistance to an antibiotic even if the mechanism of resistance is not a single gene/protein or mutation. Thus, the method does not rely on knowledge of the gene (PCR) or protein (antibody) mediating the resistance.

In certain embodiments, the infectious agent comprises an indicator gene capable of expressing an indicator protein product. In some embodiments, the method may comprise detecting the indicator protein product, wherein positive detection of the indicator protein product indicates that the microorganism of interest is present in the sample and that the microorganism is resistant to the antibiotic. In some instances, the microorganism of interest is not isolated from the sample prior to testing for antibiotic resistance. In certain embodiments, the sample is an uncultured or unenriched sample. In some cases, the method of detecting antibiotic resistance can be completed within 5 hours. In some embodiments, the method comprises treatment with lysis buffer to lyse the microorganism infected with the infectious agent prior to detecting the indicator moiety.

In another aspect of the invention, the invention comprises a method of determining effective dose of an antibiotic in killing a microorganism comprising: (a) incubating each of one or more of antibiotic solutions separately with one or more samples comprising the microorganism, wherein the concentrations of the one or more of antibiotic solutions are different and define a range, (b) incubating the microorganisms in the one or more of samples with an infectious agent comprising an indicator gene, and wherein the infectious agent is specific for the microorganism of interest, and (c) detecting an indicator protein product produced by the infectious agent in the one or more of samples, wherein detection of the indicator protein product in one or more of the plurality of samples indicates the concentrations of antibiotic solutions used to treat the one or more of the one or more of samples are not effective, and the lack of detection of the indicator protein indicates the antibiotic is effective, thereby determining the effective dose of the antibiotic.

The methods disclosed herein can be used to detect whether a microorganism of interest is susceptible or resistant to an antibiotic. A particular antibiotic may be specific for the type of microorganism it kills or inhibits; the antibiotic kills or inhibits the growth of microorganisms that are sensitive to the antibiotic and does not kill or inhibit the growth of microorganisms that are resistant to the antibiotic. In some cases, a previously sensitive microbial strain may become resistant. Resistance of microorganisms to antibiotics can be mediated by a number of different mechanisms. For example, some antibiotics disturb cell wall synthesis in a microorganism; resistance against such antibiotics can be mediated by altering the target of the antibiotic, namely a cell wall protein. In some cases, bacteria create resistance to an antibiotic by producing compounds capable of inactivating the antibiotic before reaching the bacteria. For example, some bacteria produce beta-lactamase, which is capable of cleaving the beta-lactam of penicillin or/and carbapenems, thus, inactivating these antibiotics. In some cases, the antibiotic is removed from the cell before reaching the target by a specific pump. An example is the RND transporter. In some cases, some antibiotics act by binding to ribosomal RNA (rRNA) and inhibit protein biosynthesis in the microorganism. A microorganism resistant to such antibiotic may comprise a mutated rRNA having a reduced binding capability to the antibiotic but having an essentially normal function within the ribosome. In other cases, bacteria harbor a gene that is capable of conferring resistance. For example, some MRSA harbor the mecA gene. The mecA gene product is an alternative transpeptidase with a low affinity for the ring-like structure of certain antibiotics which typically bind to transpeptidases required for bacterium cell wall formation. Therefore, antibiotics, including beta-lactams, are unable to inhibit cell wall synthesis in these bacteria. Some bacteria harbor antibiotic resistance genes that are non-functional, possibly due to mutation of the gene or regulation, which may be falsely detected as antibiotic-resistant with conventional nucleic acid methods, such as PCR, but not detected by functional methods, such as plating or culturing with antibiotics or this method.

Non-limiting examples of antibiotics that can be used in the invention include aminoglycosides, carbacephems, carbapenems, cephalosporins, glycopeptides, macrolides, monobactams, penicillin, beta-lactam antibiotic, quinolones, bacitracin, sulfonamides, tetracyclines, streptogramines, chloramphenicol, clindamycin, and lincosamide, cephamycins, lincomycins, daptomycin, oxazolidinone, and glycopeptide antibiotic.

As noted herein, in certain embodiments, the invention may comprise methods of using infectious particles for detecting resistance of microorganisms to an antibiotic or, stated another way, for detecting the efficacy of an antibiotic against a microorganism. In another embodiment, the invention comprises methods for selecting an antibiotic for treatment of an infection. Additionally, the methods may comprise methods for detecting antibiotic-resistant bacteria in a sample. The methods of the invention may be embodied in a variety of ways.

The method may comprise contacting the sample comprising the microorganism with the antibiotic and an infectious agent as described above. In some embodiments, the disclosure provides a method of determining effective dose of an antibiotic in killing or inhibiting the growth of a microorganism comprising: (a) incubating each of one or more of antibiotic solutions separately with one or more samples comprising the microorganism, wherein the concentrations of the one or more of antibiotic solutions are different and define a range, (b) incubating the microorganisms in the one or more of samples with an infectious agent comprising an indicator gene, and wherein the infectious agent is specific for the microorganism of interest, and (c) detecting an indicator protein product produced by the infectious agent in the one or more of samples, wherein detection of the indicator protein product in one or more of the plurality of samples indicates the concentrations of antibiotic solutions used to treat the one or more of the one or more of samples are not effective, and the lack of detection of the indicator protein indicates the antibiotic is effective, thereby determining the effective dose of the antibiotic.

In other embodiments, the antibiotic and the infectious agent are added sequentially, e.g., the sample is contacted with the antibiotic before the sample is contacted with the infectious agent. In certain embodiments, the method may comprise incubating the sample with the antibiotic for a period time before contacting the sample with the infectious agent. The incubation time may vary depending on the nature of the antibiotic and the microorganism, for example based on the doubling time of the microorganism. In some embodiments, the incubation time is less than 24 hours, less than 18 hours, less than 12 hours, less than 6 hours, less than 5 hours, less than 4 hours, less than 3 hours, less than 2 hours, less than 1 hour, less than 45 min, or less than 30 min. The incubation time of microorganism with the infectious agent may also vary depending on the life cycle of the particular infectious agent, in some cases, the incubation time is less than 4 hours, less than 3 hours, less than 2 hours, less than 1 hour, less than 45 min, less than 30 min. Microorganisms that are resistant to the antibiotic will survive and may multiply, and the infectious agent that is specific to the microorganism will replicate resulting in production of the indicator protein product (e.g., luciferase); conversely, microorganisms that are sensitive to the antibiotic will be killed and thus the infectious agent will not replicate. Additionally, bacteriostatic antibiotics will not kill the bacteria; however, they will halt growth and/or enrichment of the bacteria. In some instances, bacteriostatic antibiotics may interfere with bacterial protein synthesis and are expected to prevent the bacteriophage from producing an indicator molecule (e.g., luciferase). The infectious agent according to this method comprises an indicator moiety, the amount of which corresponds to the amount of the microorganisms present in the sample that have been treated with the antibiotic. Accordingly, a positive detection of the indicator moiety indicates the microorganism is resistant to the antibiotic.

In some embodiments, the methods may be used to determine whether an antibiotic-resistant microorganism is present in a clinical sample. For example, the methods may be used to determine whether a patient is infected with Staphylococcus aureus that are resistant or susceptible to a particular antibiotic. A clinical sample obtained from a patient may then be incubated with an antibiotic specific for S. aureus. The sample may then be incubated with recombinant phage specific for S. aureus for a period of time. In samples with S. aureus resistant to the antibiotic, detection of the indicator protein produced by the recombinant phage will be positive. In samples with S. aureus susceptible to the antibiotic, detection of the indicator protein will be negative. In some embodiments, methods for detection of antibiotic resistance may be used to select an effective therapeutic to which the pathogenic bacterium is susceptible.

In certain embodiments the total time required for detection is less than 6.0 hours, 5.0 hours, 4.0 hours, 3.0 hours, 2.5 hours, 2.0 hours, 1.5 hours, or less than 1.0 hour. The total time required for detection will depend on the bacteria of interest, the type of phage, and antibiotic being tested.

Optionally, the method further comprises lysing the microorganism before detecting the indicator moiety. Any solution that does not affect the activity of the luciferase can be used to lyse the cells. In some cases, the lysis buffer may contain non-ionic detergents, chelating agents, enzymes or proprietary combinations of various salts and agents. Lysis buffers are also commercially available from Promega, Sigma-Aldrich, or Thermo-Fisher. Experiments suggest that infected unlysed cells may be detectable upon addition of luciferase substrate in some embodiments. Presumably, luciferase may exit cells and/or luciferase substrate may enter cells without complete cell lysis. For example, in some embodiments the substrate for the luciferase in cell-permeable (e.g., furimazine). Thus, for embodiments utilizing the spin filter system, where only luciferase released into the lysate (and not luciferase still inside intact bacteria) is analyzed in the luminometer, lysis is required for detection. However, for embodiments utilizing filter plates or 96-well plates with phage-infected sample in solution or suspension as described below, where intact and lysed cells may be directly assayed in the luminometer, lysis may not be necessary for detection. Thus, in some embodiments, the method of detecting antibiotic resistance does not involve lysing the microorganism.

A surprising aspect of embodiments of the assays is that the step of incubating the microorganism in a sample with infectious agent only needs to be long enough for a single life cycle of the infectious agent, e.g., a phage. The amplification power of using phage was previously thought to require more time, such that the phage would replicate for several cycles. A single replication of indicator phage may be sufficient to facilitate sensitive and rapid detection according to some embodiments of the present invention. Another surprising aspect of the embodiments of the assays is that high concentrations of phage utilized for infecting test samples (i.e., high MOI) have successfully achieved detection of very low numbers of antibiotic resistant target microorganisms that have been treated with antibiotic. Factors, including the burst size of the phage, can affect the number of phage life cycles, and therefore, amount of time needed for detection. Phage with a large burst size (approximately 100 PFU) may only require one cycle for detection, whereas phage with a smaller burst size (e.g., 10 PFU) may require multiple phage cycles for detection. In some embodiments, the incubation of phage with a test sample need only be long enough for a single phage life cycle. In other embodiments, the incubation of phage with a test sample is for an amount of time greater than a single life cycle.

The phage concentration for the incubating step will vary depending on the type of phage used. In some embodiments, the phage concentration for this incubating step is greater than 1.0×10⁶, 2.0×10⁶, 3.0 10⁶, 5.0×10⁶, 6.0×10⁶, 7.0×10⁶, 8.0×10⁶, 9.0×10⁶, 1.0×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2.0×10⁷, 3.0×10⁷, 4.0×10⁷, 5.0×10⁷, 6.0×10⁷, 7.0×10⁷, 8.0×10⁷, 9.0×10⁷, or 1.0×10⁸ PFU/mL. Success with such high concentrations of phage is surprising because such large numbers of phage were previously associated with “lysis from without,” which killed target cells immediately and thereby prevented generation of useful signal from earlier phage assays. It is possible that the purification of the phage stock described herein helps to alleviate this problem (e.g., purification by sucrose gradient cesium chloride isopycnic density gradient ultracentrifugation), because in addition to removing any contaminating luciferase associated with the phage, this purification may also remove ghost particles (particles that have lost DNA). The ghost particles can lyse bacterial cells via “lysis from without,” killing the cells prematurely and thereby preventing generation of indicator signal. Electron microscopy demonstrates that a crude recombinant phage lysate (i.e., before cesium chloride purification) may have greater than 50% ghosts. These ghost particles may contribute to premature death of the microorganism through the action of many phage particles puncturing the cell membrane. Thus ghost particles may have contributed to previous problems where high PFU concentrations were reported to be detrimental.

Any of the indicator moieties as described in this disclosure may be used for detecting the viability of microorganisms after antibiotic treatment, thereby detecting antibiotic resistance. In some embodiments, the indicator moiety associated with the infectious agent may be detectable during or after replication of the infectious agent. For example, as described above, in some cases, the indicator moiety may be a protein that emits an intrinsic signal, such as a fluorescent protein (e.g., green fluorescent protein or others). The indicator may generate light and/or may be detectable by a color change. In some embodiments, a luminometer may be used to detect the indicator (e.g., luciferase). However, other machines or devices may also be used. For example, a spectrophotometer, CCD camera, or CMOS camera may detect color changes and other light emissions.

In some embodiments, exposure of the sample to antibiotic may continue for 5 minutes or more and detection at various time points may be desirable for optimal sensitivity. For example, aliquots of a primary sample treated with antibiotic can be taken at different time intervals (e.g., at 5 minutes, 10 minutes, or 15 minutes). Samples from varying time interval may then be infected with phage and indicator moiety measured following the addition of substrate.

In some embodiments, detection of the signal is used to determine antibiotic resistance. In some embodiments, the signal produced by the sample is compared to an experimentally determined value. In further embodiments, the experimentally determined value is a signal produced by a control sample. In some embodiments, the background threshold value is determined using a control without microorganisms. In some embodiments, a control without phage or without antibiotic, or other control samples may also be used to determine an appropriate threshold value. In some embodiments, the experimentally determined value is a background threshold value calculated from an average background signal plus standard deviation of 1-3 times the average background signal, or greater. In some embodiments, the background threshold value may be calculated from average background signal plus standard deviation of 2 times the average background signal. In other embodiments, the background threshold value may be calculated from the average background signal times some multiple (e.g., 2 or 3). Detection of a sample signal greater than the background threshold value indicates the presence of one or more antibiotic-resistant microorganisms in the sample. For example, the average background signal may be 250 RLU. The threshold background value may be calculated by multiplying the average background signal (e.g., 250) by 3 to calculate a value of 750 RLU. Samples with bacteria having a signal value greater than 750 RLU are determined to be positive for containing antibiotic-resistant bacteria.

Alternatively, the experimentally determined value is the signal produced by a control sample. Assays may include various appropriate control samples. For example, samples containing no infectious agent that is specific to the microorganism, or samples containing infectious agents but without microorganism, may be assayed as controls for background signal levels. In some cases, samples containing the microorganisms that have not been treated with the antibiotic, are assayed as controls for determining antibiotic resistance using the infectious agents.

In some embodiments, the sample signal is compared to the control signal to determine whether antibiotic-resistant microorganisms are present in the sample. Unchanged detection of the signal as compared to a control sample that is contacted with the infectious agent but not with the antibiotic indicates the microorganism is resistant to the antibiotic, and reduced detection of the indicator moiety as compared to a control sample that is contacted with infectious agent but not with antibiotic indicates the microorganism is susceptible to the antibiotic. Unchanged detection refers to the detected signal from a sample that has been treated with the antibiotic and infectious agent is at least 80%, at least 90%, or at least 95% of signal from a control sample that has not been treated with the antibiotic. Reduced detection refers to the detected signal from a sample that has been treated with the antibiotic and infectious agent is less than 80%, less than 70%, less than 60%, less than 50%, less than 40%, or at least 30% of signal from a control sample that has not been treated with the antibiotic.

Optionally, the sample comprising the microorganism of interest is an uncultured sample. Optionally, the infectious agent is a phage and comprises an indicator gene inserted into a late gene region of the phage such that expression of the indicator gene during phage replication following infection of host bacteria results in a soluble indicator protein product. Features of each of the compositions used in the methods, as described above, can be also be utilized in the methods for detecting antibiotic resistance of the microorganism of interest. In some embodiments, transcription of the indicator gene is controlled by the additional bacteriophage late promoter.

Also provided herein is a method of determining the effective dose of an antibiotic for killing a microorganism. In some embodiments, the antibiotic is effective at killing Staphylococcus species. For example, the antibiotic may be cefoxitin, which is effective against most methicillin-sensitive S. aureus (MSSA). Typically, one or more antibiotic solutions having different concentrations are prepared such that the different concentrations of the solutions define a range. In some cases, the concentration ratio of the least concentrated antibiotic solution to the most concentrated antibiotic solution ranges from 1:2 to 1:50, e.g., from 1:5 to 1:30, or from 1:10 to 1:20. In some cases, the lowest concentration of the one or more antibiotic solution is at least 1 μg/mL, e.g., at least 2 μg/mL, at least 5 μg/mL at least 10 μg/mL, at least 20 μg/mL, at least 40 μg/mL, at least 80 μg/mL, or at least 100 μg/mL. Each of the one or more antibiotic solutions is incubated with one aliquot of the sample comprising the microorganism of interest. In some cases, the infectious agent (e.g., bacteriophage) that is specific to the microorganism is added simultaneously with the antibiotic solutions. In some cases, the aliquots of sample are incubated with the antibiotic solutions for a period of time before the addition of the infectious agent. The indicator protein product can be detected, and positive detection indicates that the antibiotic solution is not effective and negative detection indicates the antibiotic solution is effective. The concentration of the antibiotic solution is expected to correlate to an effective clinical dose. Accordingly, in some embodiments, the method of determining effective dose of an antibiotic in killing a microorganism of interest comprises incubating each of one or more antibiotic solutions separately with a microorganism of interest in a sample, wherein the concentrations of the one or more antibiotic solutions are different and define a range; incubating the microorganism in the one or more samples with an infectious agent comprising an indicator moiety; detecting the indicator protein product of the infectious agent in the one or more samples, wherein positive detection of the indicator protein product in one or more of the one or more samples indicates the concentrations of antibiotic solutions used to treat the one or more of the one or more samples are not effective, and the lack of detection of the indicator protein indicates the antibiotic is effective, thereby determining the effective dose of the antibiotic.

In some embodiments, the method allows for determination of categorical assignment for antibiotic resistance. For example, the method disclosed herein may be used to determine the categorical assignment (e.g., susceptible, intermediate, and resistant) of an antibiotic. Susceptible antibiotics are those that are likely, but not guaranteed to inhibit the pathogenic microbe; may be an appropriate choice for treatment. Intermediate antibiotics are those that may be effective at a higher dosage, or more frequent dosage, or effective only in specific body sites where the antibiotic penetrates to provide adequate concentrations. Resistant antibiotics are those that are not effective at inhibiting the growth of the organism in a laboratory test; may not be an appropriate choice for treatment. In some embodiments, two or more antibiotic solutions are tested and the concentration ratio of the least concentrated solution and the most concentrated solution in the one or more antibiotic solutions ranges from 1:2 to 1:50, e.g., from 1:5 to 1:30, or from 1:10 to 1:20. In some cases, the lowest concentration of the one or more antibiotic solution is at least 1 μg/mL, e.g., at least 2 μg/mL, at least 5 μg/mL at least 10 μg/mL, at least 20 μg/mL, at least 40 μg/mL, at least 80 μg/mL, or at least 100 μg/mL.

In some embodiments, the present invention comprises methods for detecting antibiotic-resistant microorganisms in the presence of antibiotic-sensitive microorganisms. In certain instances, detection of antibiotic-resistant bacteria can be used to prevent the spread of infection in healthcare settings. In some embodiments, patients in a healthcare setting may be monitored for colonization of antibiotic-resistant bacteria. Preventative measures may then be implemented to prevent the spread of antibiotic-resistant bacteria.

In some embodiments of methods for detecting antibiotic resistant microorganisms, samples may contain both antibiotic-resistant and antibiotic-sensitive bacteria. For example, samples may comprise both MRSA and MSSA. In some embodiments, MRSA can be detected in the presence of MSSA without the need for isolation of MRSA from the sample. In the presence of antibiotic, MSSA does not generate a signal above the threshold value, but MRSA present in the sample are capable of producing a signal above the threshold value. Thus, if both are present within a sample, a signal above the threshold value indicates the presence of an antibiotic-resistant strain (e.g. MRSA).

In contrast to many assays known in the art, detection of antibiotic resistance of a microorganism can be achieved without prior isolation. Many methods require that a patient sample is cultured beforehand to purify/isolate individual colonies of the bacterium on an agar plate. The increased sensitivity of the methods disclosed herein, is due in part to the ability of a large number of specific infectious agents, e.g., phages to bind to a single microorganism. Following infection and replication of the phage, target microorganisms may be detected via an indicator protein product produced during phage replication.

Thus, in certain embodiments, the method may detect antibiotic resistance of a microorganism in a sample that comprises <10 cells of the microorganism (i.e., 1, 2, 3, 4, 5, 6, 7, 8, 9, 10 microorganisms). In certain embodiments, the recombinant phage can be used to detect antibiotic resistance by detection of a single bacterium of the specific type in the sample that has been treated with the antibiotic. In certain embodiments, the recombinant phage detects the presence of as few as 2, 3, 4, 5, 6, 7, 8, 9, 10, 15, 20, 30, 40, 50, 60, 70, 80, 90, or 100 of the specific bacteria in the sample that has been contacted with antibiotic.

The sensitivity of the method of detecting antibiotic resistance as disclosed herein may be further increased by washing the captured and infected microorganisms prior to incubation with the antibiotic. Isolation of target bacteria may be required when the antibiotic being assessed is known to be degraded by other bacterial species. For example, penicillin resistance would be difficult to assess without purification, since other bacteria present in a clinical sample could degrade the antibiotic (beta-lactamase secretion) and lead to a false positive. Additionally, captured microorganisms may be washed following incubation with antibiotic and the infectious agent, prior to addition of lysis buffer and substrate. These additional washing steps aid in the removal of excess parental phage and/or luciferase or other reporter protein contaminating the phage preparation. Accordingly, in some embodiments, the method of the detecting antibiotic resistance may comprise washing the captured and infected microorganisms, after adding the phage but before incubating.

In many embodiments, multi-well plates are used to conduct the assays. The choice of plates (or any other container in which detecting may be performed) may affect the detecting step. For example, some plates may include a colored or white background, which may affect the detection of light emissions. Generally speaking, white plates have higher sensitivity but also yield a higher background signal. Other colors of plates may generate lower background signal but also have a slightly lower sensitivity. Additionally, one reason for background signal is the leakage of light from one well to another, adjacent well. There are some plates that have white wells but the rest of the plate is black. This allows for a high signal inside the well but prevents well-to-well light leakage and thus may decrease background. Thus, the choice of plate or other assay vessel may influence the sensitivity and background signal for the assay.

Thus, some embodiments of the present invention solve a need by using infectious agent-based methods for amplifying a detectable signal, thereby indicating whether a microorganism is resistant to an antibiotic. The invention allows a user to detect antibiotic resistance of a microorganism that is present in a sample has not been purified or isolated. In certain embodiments as little as a single bacterium is detected. This principle allows amplification of indicator signal from one or a few cells based on specific recognition of microorganism surface receptors. For example, by exposing even a single cell of a microorganism to a plurality of phage, thereafter allowing amplification of the phage and high-level expression of an encoded indicator gene product during replication, the indicator signal is amplified such that the single microorganism is detectable. The present invention excels as a rapid test for the detection of microorganisms by not requiring isolation of the microorganisms prior to detection. In some embodiments detection is possible within 1-2 replication cycles of the phage or virus.

In additional embodiments, the disclosure comprises systems (e.g., computer systems, automated systems or kits) comprising components for performing the methods disclosed herein, and/or using the modified infectious agents described herein.

### Systems and Kits of the Invention

In some embodiments, the disclosure comprises systems (e.g., automated systems or kits) comprising components for performing the methods disclosed herein. In some embodiments, indicator phage are comprised in systems or kits according to the invention. Methods described herein may also utilize such indicator phage systems or kits. Some embodiments described herein are particularly suitable for automation and/or kits, given the minimal amount of reagents and materials required to perform the methods. In certain embodiments, each of the components of a kit may comprise a self-contained unit that is deliverable from a first site to a second site.

In some embodiments, the disclosure comprises systems or kits for rapid detection of a microorganism of interest in a sample. The systems or kits may in certain embodiments comprise a component for incubating the sample with a recombinant bacteriophage specific for the microorganism of interest, wherein the recombinant bacteriophage comprises a genetic construct, and wherein the genetic construct comprises a gene encoding an indicator protein product; and a component for detecting the indicator protein product. Some systems further comprise a component for capturing the microorganism of interest on a solid support.

In other embodiments, the disclosure comprises a method, system, or kit for rapid detection of a microorganism of interest in a sample, comprising a recombinant bacteriophage component that is specific for the microorganism of interest, wherein the recombinant bacteriophage comprises a genetic construct, and wherein the genetic construct comprises a gene encoding an indicator protein product; and a component for detecting the indicator protein product. In certain embodiments, the recombinant bacteriophage is highly specific for a particular bacterium. In an embodiment, the recombinant bacteriophage can distinguish the bacterium of interest in the presence of more than 100 other types of bacteria. In certain embodiments, a system or kit detects a single bacterium of the specific type in the sample. In certain embodiments, a system or kit detects as few as 2, 3, 4, 5, 6, 7, 8, 9, 10, 15, 20, 30, 40, 50, 60, 70, 80, 90, or 100 specific bacteria in the sample.

In certain embodiments, the systems and/or kits may further comprise a component for washing the captured microorganism sample. Additionally or alternatively, the systems and/or kits may further comprise a component for determining amount of the indicator protein product, wherein the amount of indicator moiety detected corresponds to the amount of microorganism in the sample. For example, in certain embodiments, the system or kit may comprise a luminometer or other device for measuring a luciferase enzyme activity.

In some systems and/or kits, the same component may be used for multiple steps. In some systems and/or kits, the steps are automated or controlled by the user via computer input and/or wherein a liquid-handling robot performs at least one step.

Thus in certain embodiments, the invention may comprise a system or kit for rapid detection of a microorganism of interest in a sample, comprising: a component for incubating the sample with a recombinant bacteriophage specific for the microorganism of interest, wherein the recombinant bacteriophage comprises a gene encoding an indicator protein product; a component for capturing the microorganism from the sample on a solid support; a component for washing the captured microorganism sample to remove unbound infectious agent; and a component for detecting the indicator protein product. In some embodiments, the same component may be used for steps of capturing and/or incubating and/or washing (e.g., a filter component). Some embodiments additionally comprise a component for determining the amount of the microorganism of interest in the sample, wherein the amount of indicator protein product detected corresponds to the amount of microorganism in the sample. Such systems can include various embodiments and subembodiments analogous to those described above for methods of rapid detection of microorganisms. In an embodiment, the microorganism is a bacterium and the infectious agent is a bacteriophage. In a computerized system, the system may be fully automated, semi-automated, or directed by the user through a computer (or some combination thereof).

In an embodiment, the disclosure comprises a system or kit comprising components for detecting a microorganism of interest comprising: a component for infecting the at least one microorganism with a plurality of recombinant bacteriophages; a component for lysing the at least one infected microorganism; and a component for detecting the soluble indicator protein product encoded and expressed by the recombinant bacteriophage, wherein detection of the soluble protein product of the infectious agent indicates that the microorganism is present in the sample.

In some embodiments, the disclosure comprises a system or kit comprising components for treating a biofilm-related infection comprising: a component for

These systems and kits of the disclosure include various components. As used herein, the term “component” is broadly defined and includes any suitable apparatus or collections of apparatuses suitable for carrying out the recited method. The components need not be integrally connected or situated with respect to each other in any particular way. The invention includes any suitable arrangements of the components with respect to each other. For example, the components need not be in the same room. But in some embodiments, the components are connected to each other in an integral unit. In some embodiments, the same components may perform multiple functions.

### Computer Systems and Computer Readable Media

The system, as described in the present technique or any of its components, may be embodied in the form of a computer system. Typical examples of a computer system include a general-purpose computer, a programmed microprocessor, a microcontroller, a peripheral integrated circuit element, and other devices or arrangements of devices that are capable of implementing the steps that constitute the method of the present technique.

A computer system may comprise a computer, an input device, a display unit, and/or the Internet. The computer may further comprise a microprocessor. The microprocessor may be connected to a communication bus. The computer may also include a memory. The memory may include random access memory (RAM) and read only memory (ROM). The computer system may further comprise a storage device. The storage device can be a hard disk drive or a removable storage drive such as a floppy disk drive, optical disk drive, etc. The storage device can also be other similar means for loading computer programs or other instructions into the computer system. The computer system may also include a communication unit. The communication unit allows the computer to connect to other databases and the Internet through an I/O interface. The communication unit allows the transfer to, as well as reception of data from, other databases. The communication unit may include a modem, an Ethernet card, or any similar device which enables the computer system to connect to databases and networks such as LAN, MAN, WAN and the Internet. The computer system thus may facilitate inputs from a user through input device, accessible to the system through I/O interface.

A computing device typically will include an operating system that provides executable program instructions for the general administration and operation of that computing device, and typically will include a computer-readable storage medium (e.g., a hard disk, random access memory, read only memory, etc.) storing instructions that, when executed by a processor of the server, allow the computing device to perform its intended functions. Suitable implementations for the operating system and general functionality of the computing device are known or commercially available, and are readily implemented by persons having ordinary skill in the art, particularly in light of the disclosure herein.

The computer system executes a set of instructions that are stored in one or more storage elements, in order to process input data. The storage elements may also hold data or other information as desired. The storage element may be in the form of an information source or a physical memory element present in the processing machine.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (“SAN”) familiar to those skilled in the art. Similarly, any necessary files for performing the functions attributed to the computers, servers, or other network devices may be stored locally and/or remotely, as appropriate. Where a system includes computing devices, each such device can include hardware elements that may be electrically coupled via a bus, the elements including, for example, at least one central processing unit (CPU), at least one input device (e.g., a mouse, keyboard, controller, touch screen, or keypad), and at least one output device (e.g., a display device, printer, or speaker). Such a system may also include one or more storage devices, such as disk drives, optical storage devices, and solid-state storage devices such as random access memory (“RAM”) or read-only memory (“ROM”), as well as removable media devices, memory cards, flash cards, etc.

Such devices also can include a computer-readable storage media reader, a communications device (e.g., a modem, a network card (wireless or wired), an infrared communication device, etc.), and working memory as described above. The computer-readable storage media reader can be connected with, or configured to receive, a computer-readable storage medium, representing remote, local, fixed, and/or removable storage devices as well as storage media for temporarily and/or more permanently containing, storing, transmitting, and retrieving computer-readable information. The system and various devices also typically will include a number of software applications, modules, services, or other elements located within at least one working memory device, including an operating system and application programs, such as a client application or Web browser. It should be appreciated that alternate embodiments may have numerous variations from that described above. For example, customized hardware might also be used and/or particular elements might be implemented in hardware, software (including portable software, such as applets), or both. Further, connection to other computing devices such as network input/output devices may be employed.

Non-transient storage media and computer readable media for containing code, or portions of code, can include any appropriate media known or used in the art, including storage media and communication media, such as but not limited to volatile and non-volatile, removable and non-removable media implemented in any method or technology for storage and/or transmission of information such as computer readable instructions, data structures, program modules, or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices, or any other medium which can be used to store the desired information and which can be accessed by the a system device. Based on the disclosure and teachings provided herein, a person of ordinary skill in the art will appreciate other ways and/or methods to implement the various embodiments.

A computer-readable medium may comprise, but is not limited to, an electronic, optical, magnetic, or other storage device capable of providing a processor with computer-readable instructions. Other examples include, but are not limited to, a floppy disk, CD-ROM, DVD, magnetic disk, memory chip, ROM, RAM, SRAM, DRAM, content-addressable memory (“CAM”), DDR, flash memory such as NAND flash or NOR flash, an ASIC, a configured processor, optical storage, magnetic tape or other magnetic storage, or any other medium from which a computer processor can read instructions. In one embodiment, the computing device may comprise a single type of computer-readable medium such as random access memory (RAM). In other embodiments, the computing device may comprise two or more types of computer-readable medium such as random access memory (RAM), a disk drive, and cache. The computing device may be in communication with one or more external computer-readable mediums such as an external hard disk drive or an external DVD or Blu-Ray drive.

As discussed above, the embodiment comprises a processor which is configured to execute computer-executable program instructions and/or to access information stored in memory. The instructions may comprise processor-specific instructions generated by a compiler and/or an interpreter from code written in any suitable computer-programming language including, for example, C, C++, C#, Visual Basic, Java, Python, Perl, JavaScript, and ActionScript (Adobe Systems, Mountain View, Calif.). In an embodiment, the computing device comprises a single processor. In other embodiments, the device comprises two or more processors. Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

The computing device comprises a network interface. In some embodiments, the network interface is configured for communicating via wired or wireless communication links. For example, the network interface may allow for communication over networks via Ethernet, IEEE 802.11 (Wi-Fi), 802.16 (Wi-Max), Bluetooth, infrared, etc. As another example, network interface may allow for communication over networks such as CDMA, GSM, UMTS, or other cellular communication networks. In some embodiments, the network interface may allow for point-to-point connections with another device, such as via the Universal Serial Bus (USB), 1394 FireWire, serial or parallel connections, or similar interfaces. Some embodiments of suitable computing devices may comprise two or more network interfaces for communication over one or more networks. In some embodiments, the computing device may include a data store in addition to or in place of a network interface.

Some embodiments of suitable computing devices may comprise or be in communication with a number of external or internal devices such as a mouse, a CD-ROM, DVD, a keyboard, a display, audio speakers, one or more microphones, or any other input or output devices. For example, the computing device may be in communication with various user interface devices and a display. The display may use any suitable technology including, but not limited to, LCD, LED, CRT, and the like.

The set of instructions for execution by the computer system may include various commands that instruct the processing machine to perform specific tasks such as the steps that constitute the method of the present technique. The set of instructions may be in the form of a software program. Further, the software may be in the form of a collection of separate programs, a program module with a larger program or a portion of a program module, as in the present technique. The software may also include modular programming in the form of object-oriented programming. The processing of input data by the processing machine may be in response to user commands, results of previous processing, or a request made by another processing machine.

While the present invention has been disclosed with references to certain embodiments, numerous modifications, alterations and changes to the described embodiments are possible without departing from the scope and spirit of the present invention, as defined in the appended claims. Accordingly, it is intended that the present invention not be limited to the described embodiments, but that it have the full scope defined by the language of the following claims, and equivalents thereof.

### EXAMPLES

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter.

### Example 1. Staphylococcus aureus Biofilm and Irrigation Wash Testing Protocol

Overnight cultures of S. aureus were diluted into either 200 μL TSB (100% Tryptone Soya Broth), TSBg (66% TSB+0.2% glucose), or TSB-HS (90% TSB+10% human serum). The initial inoculum was a 200-fold dilution of overnight culture and prepared in 96-well plates. Plates were covered and incubated statically at 37° C. for at least 16 hours. Non-biofilm planktonic cells were removed by discarding the media and washing gently with 200 μL saline. Saline irrigation wash was performed by pipetting 200 μL of saline forcibly onto the biofilm. This direct wash is expected to mechanically release portions of the adherent biofilm. 150 μL of each saline irrigation wash sample was transferred to a separate 96-well plate containing dried-down concentrated BHI (Brain Heart Infusion). The final concentration of BHI in each well was 1× (37 g/L). In order to assess the residual adherent biomass, 150 μL of BHI was added to each well containing the post-irrigation washed biofilm. All samples (residual biofilm+saline irrigation wash) were covered and incubated statically at 37° C. to facilitate enrichment over a four-hour period. After enrichment, 10 μL of a recombinant phage cocktail was added to each well and mixed by pipetting. Plates were once again covered and incubated statically at 37° C. for two hours. After infection, 65 μL of detection master mix containing NANO-GLO® buffer, NANO-GLO® substrate and Renilla lysis buffer was added to each well and mixed again by pipetting. Samples were read on a GLOMAX® luminometer after a 3-minute wait time and utilizing a 1 second integration. Data is presented as relative light unit (RLUs) in Table 1.

TABLE 1
Phage Detection of S. aureus

| Saline Irrigation | | RLU Signal (Positive | | |
|---|---|---|---|---|
| Wash of Biofilm | | cutoff is 3x Background) | | |
| ATCC | | TSB | TSBg | TSB-HS |
| S. aureus Strain | Type | Biofilm | Biofilm | Biofilm |
| BAA-1763 | MRSA | 387000 | 3336000 | 984800 |
| BAA-1768 | MRSA | 611900 | 9260000 | 2628000 |
| BAA-42 | MRSA | 35890 | 120500 | 18010 |
| BAA-1720 | MRSA | 7652000 | 11030000 | 27120000 |
| 33592 | MRSA | 401700 | 1041000 | 4530000 |
| 12600 | MSSA | 998000 | 9317000 | 14800000 |
| Background | Saline | 335 | 395 | 371 |
| Control | | | | |
| Post-Wash | | RLU Signal (Positive | | |
| Residual Biofilm | | cutoff is 3x Background) | | |
| ATCC | | TSB | TSBg | TSB-HS |
| S. aureus Strain | Type | Biofilm | Biofilm | Biofilm |
| BAA-1763 | MRSA | 171600 | 2772000 | 114300 |
| BAA-1768 | MRSA | 262400 | 2304000 | 117300 |
| BAA-42 | MRSA | 18990 | 78880 | 8318 |
| BAA-1720 | MRSA | 2854000 | 9420000 | 13000000 |
| 33592 | MRSA | 581200 | 919900 | 1010000 |
| 12600 | MSSA | 211500 | 35220000 | 308700 |
| Background | BHI | 150 | 173 | 152 |
| Control | | | | |

TSB-100% TSB
TSBg-66% TSB + 0.2% glucose
TSB-HS-90% TSB + 10% Human serum

We claim:

1. A method for the diagnosis and treatment of a biofilm-related infection in a subject comprising the steps of:
(i) providing a biological sample taken from the subject;
(ii) diagnosing the subject with a biofilm-related infection by detecting the presence of at least one microorganism of interest comprising the steps of:
(a) contacting at least one aliquot of the biological sample with an amount of a reporter cocktail composition comprising at least one recombinant bacteriophage;
(b) detecting a signal produced following replication of the recombinant bacteriophage, wherein detection of the signal indicates the microorganism of interest is present in the sample; and
(iii) treating the subject diagnosed with a biofilm-related infection comprising the steps of:
(a) selecting a therapeutic cocktail composition based on the diagnosis of step (ii);
(b) administering a therapeutically-effective amount of a therapeutic cocktail composition comprising at least one bacteriophage, wherein the bacteriophage is specific for the detected microorganism of interest; and
(c) optionally administering at least one additional therapeutic agent.

2. The method of claim 1, wherein the biofilm is on or around the surface of an implant in a subject suspected of having an infection.

3. The method of claim 1, wherein the step of diagnosing the subject comprises contacting a plurality of aliquots with a plurality of reporter cocktail compositions.

4. The method of claim 1 wherein the step of diagnosing the subject further comprises determining the antibiotic resistance of the detected microorganism of interest.

5. The method of claim 4, wherein determining the antibiotic resistance of the detected microorganism of interest further comprises the step of contacting the biological sample with an antibiotic prior to contacting the biological sample with the reporter cocktail composition.

6. The method of claim 1, wherein the recombinant bacteriophage of the reporter cocktail composition comprises a genetic construct inserted into a bacteriophage genome, wherein the genetic construct comprises an indicator gene and an additional bacteriophage late promoter.

7. The method of claim 6, wherein the indicator gene does not encode a fusion protein and transcription of the indicator gene is controlled by the additional bacteriophage late promoter.

8. The method of claim 7, wherein expression of the indicator gene during bacteriophage replication following infection of a host bacterium results in the indicator protein product.

9. The method of claim 8, wherein the indicator gene encodes a luciferase enzyme.

10. The method of claim 1, wherein the bacteriophage of the therapeutic cocktail composition is a recombinant bacteriophage.

11. The method of claim 10, wherein the recombinant bacteriophage of the therapeutic cocktail composition comprises a genetic construct inserted into a bacteriophage genome, wherein the genetic construct comprises an enzyme.

12. The method of claim 11, wherein the enzyme is a glycosidase, amidase, or an endopeptidase.

13. The method of claim 1, wherein the microorganism of interest is Staphylococcus species, Klebsiella species, Pseudomonas species, Cutibacterium acnes, and Shigella species.

14. The method of claim 1, wherein at least one type of recombinant bacteriophage is constructed from Phage K, MP115, or ISP.

15. The method of claim 1, wherein the biological sample is first incubated in conditions favoring growth for an enrichment period of 24 hours, 23 hours, 22 hours, 21 hours, 20 hours, 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, or 2 hours.

16. The method of claim 1, wherein the therapeutic agent is an antibiotic.

17. A method of preventing or inhibiting infection in a subject comprising applying a cocktail composition comprising at least one recombinant bacteriophage to a surgical implant, dressing, or suture.

18. A surgical implant, dressing, or suture coated in a cocktail composition comprising at least one recombinant bacteriophage.
